(19) United States
(12) Patent Application Publication
(10) Pub. No.: US 2006/0247698 A1
(43) Pub. Date: Nov. 2, 2006

(54) Multi-site PESP with fusion pacing

(76) Inventors: John E. Burnes, Andover, MN (US); Glenn C. Zillmer, Hudson, WI (US); D. Curtis Deno, Andover, MN (US)

Correspondence Address: MEDTRONIC, INC., 710 MEDTRONIC PARK, MINNEAPOLIS, MN, 55432-9924, US

(21) Appl. No.: 11/116,877

(22) Filed: Apr. 28, 2005

(51) Int. Cl. A61N 1/362

(52) U.S. Cl. 607/9

(57) ABSTRACT

In some embodiments, a method of operating an implantable cardiac pacing device to provide coupled ventricular pacing may include one or more of the following steps: (a) sensing ventricular events at a first ventricular site and generating a ventricular sense event signal in response thereto, (b) providing coupled pacing pulses simultaneously at the first ventricular site and at a second ventricular site at a ventricular extra stimulus interval (VESI) timed from immediately preceding ventricular sense event signals sufficient to effect post-extra-systolic potentiation (PESP) of the ventricular sites, and (c) providing pacing pulse at the second ventricular site after sensing ventricular events at the first ventricular site.

### FIELD

The embodiments of the invention relate to the application of paired and/or coupled pacing stimulation to effect post-extrasystolic potentiation (PESP) cardiac output augmentation.

### BACKGROUND

Atrioventricular (AV) synchronous pacing systems, including DDD pacing systems marketed by Medtronic, Inc. and other companies, have been prescribed for treatment of a variety of bradycardia and/or congestive heart failure conditions in patients. Patients with bradycardia or AV block tend to improve with bradycardia pacing systems that may include features such as AV synchrony and physiologic sensor driven rate response. However, in some patients bradycardia pacing does not always lead to improvement in cardiac output and alleviation of the symptoms attendant to progressive cardiovascular disease processes. Patients with heart failure and ventricular dysynchrony can improve their heart failure status through cardiac resynchronization therapy (CRT). Several forms of heart failure are also associated with compromised diastolic function and/or decreased atrial and ventricular compliance. These may be conditions associated with damage from myocardial infarction, idiopathic cardiomyopathies, chronic disease processes or complications from cardiac surgery with or without specific disease processes. Most heart failure patients do not normally suffer from a defect in the conduction system leading to ventricular bradycardia, but rather suffer from symptoms which may include a general weakening of the contractility of the cardiac muscle, attendant enlargement thereof, depressed ventricular filling characteristics, edema, and disruption in systemic blood pressure. All these disease processes lead to insufficient cardiac output to sustain even moderate levels of exercise and proper function of other body organs. Such patients are normally treated with drug therapies, including beta-blockers, ace inhibitors, or digitalis, which may only slow the heart failure disease process or even lead to toxicity and loss of effectiveness.

In the early days of implantable cardiac pacing, it was observed that paired and triggered (also referred to as coupled) pacing with relative short interpulse intervals (150 to 250 milliseconds in dogs and about 300 milliseconds in human subjects) results in electrical depolarizations without attendant mechanical myocardial contractions. The result of the second pulse, applied within the relative refractory period of the first paced or spontaneous depolarization, is to prolong the refractory period and effectively slow the mechanical heart rate from its spontaneous rhythm. This slowing effect has been employed since that time in many applications, including the treatment of atrial and ventricular tachycardias, where a single pulse or a burst of pulses are coupled to a spontaneous tachycardia event with a coupling interval that is shorter than and can be set as a fraction of the tachycardia interval as taught, for example, in U.S. Pat. No. 3,857,399 to Dr. Fred Zacouto and U.S. Pat. No. 3,939,844 to Michael Pequiguot. The slowing of the heart rate by coupled pacing is accompanied by the ability to increase or decrease the rate with subsequent paired pacing within wide limits.

Paired and coupled stimulation also causes an augmentation of contractile force effect through a phenomenon known as post-extrasystolic potentiation. The effect can be performed continuously provided there is a continuous string of extrasystoles. When removed, the effect decays over the next few contractions until the baseline levels of force production are reached. The extent of the augmentation is closely related to the prematurity of the extrasystole, the extra-systolic interval (ESI).

Early investigators conducted a large number of animal and human studies employing paired and coupled stimulation of the atrial and ventricular chambers in an effort to employ the PESP effect for the ventricles therapeutically. A history of the investigations and studies conducted in the 1960's is published in the book Cardiac Pacemakers by Harold Siddons and Edgar Sowton, M.D., 1968, pages 201-216 and the bibliography listing articles referenced therein. In addition, medical device manufacturers, including Medtronic, Inc., offered paired and coupled pacing pulse stimulators over many years to investigators conducting such studies. The Medtronic.RTM. Model 5837 R-wave coupled pulse generator is an example of such non-implanted pulse generators which were used by investigators to conduct paired and coupled pacing studies where both the pacing rate and the coupling intervals were manually adjustable.

In the studies conducted with such systems, and as reported in the above-referenced Siddons et al. book and papers referenced therein, it was also observed that PESP effect is more marked in animals and patients when myocardial function is poor rather than normal. It was also observed that the “electro-augmentation” of the force of contraction provided by the PESP effect is not increased by a third electrical stimulus. Thus, usually only a second pacing pulse, either paired with a preceding pacing pulse or as triggered by a preceding spontaneous cardiac event, was employed in further studies. Such studies have included the delivery of paired or triggered pacing pulses to either the ventricle or the atrium. It was observed that in those patients that have normal AV conduction, the ventricular rate could be slowed by paired or coupled stimulation of the atrium. However, the ventricular contraction was not found to be electro-augmented by such atrial stimulation.

Other physiologic effects of the paired and coupled pacing included in the PESP effects described above attendant changes in the contractile force of the myocardium are the peak systolic blood pressure, the rate of contraction of the ventricular muscle with a resulting increase of the rate of rise of intraventricular pressure (dP/dt), an increase in coronary blood flow, and an increase in the oxygen uptake of the heart per beat. Investigators observed that PESP was accompanied by an increase in the myocardial oxygen consumption of 35% to 70% as compared with single pulse stimulation at the same rate. The addition of a third stimulus increased the myocardial oxygen uptake even further without any attendant observed increase in cardiac contractile force. The alterations in coronary flow roughly parallel the oxygen consumption of the heart as observed in such studies.

The marked augmentation effect produced by paired stimulation led certain investigators to study the use of the technique in the treatment of acute heart failure induced in dogs. Improvements in left ventricular performance and cardiac output produced by such paired pacing in these dogs was observed by several investigators. In other studies conducted on relatively normal dogs' hearts, it was confirmed that paired pacing offered no increase in cardiac output, most likely due to reflex compensation.

Delivery of an extrasystole at a single site may result in slowed wavefront propagation around the heart due to the ectopic origin of the paced extrasystole (cell to cell conduction) and slowed myocardial recovery associated with propagation of an extrasystole. This may result in the need for longer effective ESIs (Extra Stimulus Interval) at sites distant to where the therapy is delivered to achieve a maximal PESP effect. Therefore, single site PESP may not provide optimal therapy because of a loss of augmentation at sites that are activated later.

Various stimulation regimens have been proposed for the treatment of heart failure which involve application of supra-threshold and/or sub-threshold stimulation paired or coupled pacing pulses or pulse trains. Moreover, various electrodes have been proposed for single site and multi-site delivery of the stimulation pulses to one or more heart chambers in the above-referenced patents and publications.

U.S. Pat. No. 5,213,098 discloses PESP cardiac pacing energy stimulator for applying paired and/or triggered pacing stimulation pulses to the right atrium and/or ventricle incorporating one or more sensors and signal processing circuitry for controlling the frequency of or number of heart cycles between periodic delivery of triggered or paired pacing to induce and optimize the PESP effect for the treatment of CHF or other cardiac dysfunctions. A first sensor, e.g., a ventricular or arterial blood pressure or flow sensor, is employed to monitor the performance of the heart and to develop a cardiac performance index (CPI). A second sensor, e.g., an oxygen saturation sensor positioned in the coronary sinus, is employed to monitor cardiac muscle stress and develop a cardiac stress index (CSI) to balance performance and stress. The disclosed PESP stimulator may be incorporated into a dual chamber (DDD) pacing system with or without physiologic rate control and with or without backup cardioversion/defibrillation therapy capabilities or in a separate, single purpose device. Atrial PESP stimulation has particular application in augmenting filling of the ventricles.

A series of PCT publications including, for example, PCT WO 97/25098 describe the application of one or more “non-excitatory” anodal or cathodal stimulation pulses to the heart and maintain that improvements in LV performance may be realized without capturing the heart. In a further commonly assigned U.S. Pat. No. 5,800,464, sub-threshold anodal stimulation is provided to the heart to condition the heart to mechanically respond more vigorously to the conventional cathodal supra-threshold pacing pulses.

Mechanical function is strongly related to synchronous contraction and to well timed ventricular PESP stimulation. Multi-site pacing (usually in the RV and LV) has been employed in cardiac resynchronization therapy (CRT) for heart failure and proposed for PESP therapy implementations to address conduction delays and dyssynchrony. There are, however, dual goals for a combined stimulation therapy: 1) employing RV and/or LV pacing pulses for the initial S1 systole to produce a maximally synchronous mechanical contraction, and 2) providing RV and/or LV pulses timed for the S2 extrasystole to maximize PESP potentiation in each respective chamber and the whole heart. Because of conduction delays or other causes of dyssynchrony, stimulation timing to achieve both goals may require fusion or triggered pulses at multiple sites to pace and/or potentiate. However, optimal therapy timing may vary with time and physiologic state. It would be helpful to provide improved means to periodically assess the physiologic milieu and provide optimal therapeutic stimulation timing for subsequent cardiac cycles.

### SUMMARY

In some embodiments, a method of operating an implantable cardiac pacing device to provide coupled ventricular pacing may include one or more of the following steps: (a) sensing ventricular events at a first ventricular site and generating a ventricular sense event signal in response thereto, (b) providing coupled pacing pulses simultaneously at the first ventricular site and at a second ventricular site at a ventricular extra stimulus interval (VESI) timed from immediately preceding ventricular sense event signals sufficient to effect post-extra-systolic potentiation (PESP) of the ventricular sites, (c) providing pacing pulse at the second ventricular site after sensing ventricular events at the first ventricular site, and (d) sensing atrial events at an atrial site, providing a ventricular pacing pulse at the second ventricular site following the sensed atrial event such that depolarization waves resulting from intrinsic ventricular depolarizations at the first ventricular site and paced depolarizations at the second ventricular site fuse together at some intermediate location between the first and second ventricular sites.

In some embodiments, a method of operating an implantable cardiac pacing device to provide coupled ventricular pacing may include one or more of the following steps: (a) sensing ventricular events at a first ventricular site and generating a first ventricular sense event signal in response thereto, (b) sensing ventricular events at a second ventricular site and generating a second ventricular sense event signal in response thereto, (c) providing pacing pulses at the first ventricular site at a ventricular extra stimulus interval (VESI) timed from immediately preceding first ventricular sense event signals sufficient to effect post-extra-systolic potentiation (PESP) of the first ventricular site, (d) providing a pacing pulse at the second ventricular site at the VESI sufficient to effect PESP of the second ventricular site, (e) providing pacing pulses at the second ventricular site after sensing ventricular events at the first ventricular site, and (f) sensing atrial events at an atrial site, providing a ventricular pacing pulse at the second ventricular site following the sensed atrial event such that depolarization waves resulting from intrinsic ventricular depolarizations at the first ventricular site and paced depolarizations at the second ventricular site fuse together at some intermediate location between the first and second ventricular sites.

In some embodiments, a method of operating an implantable cardiac pacing device to provide coupled pacing may include one or more of the following steps: (a) sensing intrinsic atrial depolarizations at an atrial site and generating an atrial sense event signal in response thereto, (b) sensing intrinsic ventricular depolarizations at a first ventricular site and generating a first ventricular sense event signal in response thereto, (c) providing coupled pacing pulses at the atrial site at an atrial extra stimulus interval (AESI) timed from the atrial sense event signal sufficient to effect post-extra-systolic potentiation (PESP) of the atrium, (d) sensing intrinsic ventricular depolarizations at the first ventricular site following immediately preceding coupled pacing pulses at the atrial site and generating a second ventricular sense event signal in response thereto, (e) providing coupled pacing pulses at a second ventricular site at a ventricular extra stimulus interval (VESI) timed from one of the immediately preceding second ventricular sense event signal and the immediately preceding atrial sense event signal sufficient to effect PESP of the second ventricular site.

### DRAWINGS

FIG. 1 depicts the relationship of heart chamber EGM, pressure, flow, and volume during a cardiac cycle;

FIG. 2 is a schematic diagram depicting a multi-channel, atrial and bi-ventricular, monitoring/pacing IMD in which the embodiments of the invention is preferably implemented;

FIG. 3A is a simplified block diagram of one embodiment of IPG circuitry and associated leads employed in the system of FIG. 2 enabling therapy delivery and heart failure state monitoring in one or more heart chamber;

FIG. 3B, is a simplified block diagram of another embodiment of IPG circuitry and associated leads that can be employed in the system of FIG. 2 enabling PESP therapy delivery and heart failure state monitoring in one or more heart chambers;

FIG. 4 is a simplified block diagram of a single monitoring and pacing channel for deriving pressure, impedance and cardiac EGM signals employed in monitoring CHF and optionally pacing the heart and delivering PESP therapy in accordance with the embodiments of the invention;

FIG. 5 depicts the delivery of therapeutic PESP stimulation, particularly, pacing energy pulse trains commenced during the refractory period of the heart and continuing for a PESP delivery interval;

FIG. 6 is a set of three X-Y plots representing physiologic and therapy activity according to the embodiments of the invention;

FIG. 7 is a flow chart depicting an aspect of the embodiments of the invention;

FIG. 8 is a flow chart depicting another aspect of the embodiments of the invention;

FIG. 9 is a flow chart depicting yet another aspect of the embodiments of the invention;

FIG. 10 is a flow chart depicting an additional aspect of the embodiments of the invention;

FIG. 11 is a set of traces representing physiologic and therapy activity according to the embodiments of the invention;

FIG. 12 is a set of traces representing physiologic and therapy activity according to the embodiments of the invention;

FIG. 13 is a set of traces representing physiologic and therapy activity according to the embodiments of the invention;

FIG. 14 is a set of traces representing physiologic and therapy activity according to the embodiments of the invention;

FIG. 15 is a flow chart depicting an additional aspect of the embodiments of the invention;

FIG. 16 is a set of four X-Y plots illustrating timing relationships between stimulation amplitude, mechanical function, arrhythmia risk and “net benefit” of therapy delivery according to the embodiments of the invention;

FIG. 17 is a set of traces representing physiologic and therapy activity according to the embodiments of the invention;

FIG. 18 is a set of traces representing physiologic and therapy activity according to the embodiments of the invention;

FIG. 19 is a flow chart depicting an additional aspect of the embodiments of the invention;

FIG. 20 is a flow diagram showing programs for determining and controlling the ESI in embodiments of the invention;

FIG. 21 is a marker channel diagram of a simultaneous extrasystole to the right and left ventricle in an embodiment of the embodiments of the invention;

FIG. 22 is a marker channel diagram of a sequential extrasystole to the right and left ventricle in an embodiment of the embodiments of the invention;

FIG. 23 is a marker channel diagram of a sequential pacing of the right and left ventricle with simultaneous extrasystoles to the right and left ventricle in an embodiment of the embodiments of the invention;

FIG. 24 is a marker channel diagram of an extrasystole delivered to the right and left ventricle based upon a triggering event in an embodiment of the embodiments of the invention;

FIG. 25 is a marker channel diagram of a simultaneous extrasystole delivered to the right and left ventricle based upon a triggering event in another embodiment of the embodiments of the invention;

FIG. 26 is a marker channel diagram of a simultaneous extrasystole delivered to the right and left ventricle with fusion pacing of the ventricles in an embodiment of the embodiments of the invention;

FIG. 27 is a marker channel diagram of a sequential extrasystole delivered to the right and left ventricle with fusion pacing of the ventricles in an embodiment of the embodiments of the invention;

FIG. 28 is a marker channel diagram of an extrasystole delivered to the right and left ventricle with PESP fusion pacing of the ventricles in an embodiment of the present invention.

### DESCRIPTION OF VARIOUS EMBODIMENTS

The following discussion is presented to enable a person skilled in the art to make and use the embodiments of the invention. Various modifications to the illustrated embodiments will be readily apparent to those skilled in the art, and the generic principles herein may be applied to other embodiments and applications without departing from the embodiments of the invention. Thus, the embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of the embodiments of the invention. Skilled artisans will recognize the examples provided herein have many useful alternatives and fall within the scope of the embodiments of the invention. The following introductory material is intended to familiarize the reader with the general nature and some of the features of the embodiments of the invention.

A system constructed and operated according to the embodiments of the invention that may be used to deliver the therapies discussed above may include a signal generator, timing circuit, and/or microprocessor control circuit of the type included in existing pacemaker or ICD (implantable cardioverter defibrillator) systems as is known in the art. Exemplary systems are shown in U.S. Pat. Nos. 5,158,078, 5,318,593, 5,226,513, 5,314,448, 5,366,485, 5,713,924, 5,224,475 and 5,835,975 each of which is incorporated herein by reference, although any other type of pacing and/or ICD system may be used for this purpose. In such systems, EGM sensing is performed by electrodes carried on leads placed within the chambers of the heart, and/or on the housing of the device. Alternatively, subcutaneous and/or external pad or patch electrodes may be used to sense cardiac signals. Physiological sensors may likewise be carried on device housings or lead systems according to any of the configurations and/or sensing systems known in the art.

All embodiments of the invention share a common need for electrode configurations to deliver electrical stimulation energy where necessary and to time the delivery of this energy to achieve beneficial effects while avoiding unsafe delivery (as further described hereinbelow). For each therapy component described above, specific electrode locations and geometries may be preferred. The locations for the electrodes of these embodiments of the invention for stimulation include: use of large surface area defibrillation coil electrodes in the heart or adjacent to the heart; pacing electrodes at locations including RV apex, outflow tract, atrial locations, HIS bundle site, left side epicardium, pericardial surface of the heart or endocardium; transthoracic electrodes including paddles and patches, can electrode, temporary electrodes (e.g., epicardial, transvenous or post-operative electrodes), subcutaneous electrodes and multiple site stimulation.

In accordance with common biomedical engineering practices, stimulation therapy is applied with minimized net charge delivery to reduce corrosion and counteract polarization energy losses. Both energy efficient therapy delivery and electrogram (EGM) sensing benefit from low polarization lead systems. Finally, the electrodes are preferably connected to fast recovery amplifiers that allow EGM sensing soon after therapy delivery.

The most fundamental sensors are those based on electrograms (ECG or EGMs) and reflect cardiac electrical activity. These sensors require electrodes located where they can readily detect depolarization and repolarization signals as well as sense amplifiers for the monitoring of heart rhythm and diagnosis of arrhythmias.

According to one embodiment, blood pressure sensors, accelerometers, flow probes, microphones, or sonometric crystals may be used to measure flow, force, velocity, movement of the walls of the heart, and/or to estimate the volume of the cardiac chambers. Parameters derived from these sensors can also be used to detect the onset and severity of cardiac hemodynamic dysfunction. For example, HF decompensation may be indicated when a change in long-term diastolic cardiac pressure has increased while contractility of the heart derived from dP/dt rate of rise of ventricular pressure (dP/dtₘₐₓ) has diminished. Although pressure sensors figure prominently in the examples above a number of other sensors could reflect mechanical function. Intracardiac or transthoracic impedance changes reflect mechanical function, stroke volume, and cardiac output. Accelerometers or microphones within the body or applied externally sense serious cardiac dysfunction and monitor the response to therapy. Heart volume, dimension changes, and velocities may be measured by implanted or external applications of ultrasound.

Another embodiment of these embodiments of the invention may utilize changes in transthoracic or intracardiac impedance signals to sense cardiac motion and respiratory movement. Changes in intra-thoracic impedance as a result of pulmonary edema may also be used trigger PESP stimulation therapy.

In implantable or external devices, metabolic or chemical sensors such as expired CO₂ and blood oxygen saturation, pH, pO₂, and/or lactate) may be employed to reflect cardiac dysfunction.

Another aspect of these embodiments of the invention involves delivering electrical stimulation to the atrium and ventricles in a manner that optimizes resulting mechanical function including pressures and flows while minimizing associated risks. Several features of the embodiments of the invention are provided to achieve this goal, including regulation of PESP therapy delivery to attain the desired level of enhanced function, the use of atrial coordinated pacing, or ACP, to improve rhythm regularity and hemodynamic benefit over PESP alone, and delivery rules to inhibit or lockout PESP therapy when it is at risk of being proarrhythmic, diminishing diastole and coronary blood flow, and/or reducing the beneficial effect on hemodynamics. Rapid PESP therapy heart rates are a prime example of when PESP therapy is counter productive and may necessitate the use of such delivery lockout rules.

A delivery lockout rule operates on a short term or beat-by-beat basis to disable PESP (and ACP, if enabled) if the V-V interval from the prior cycle is too short. Thus, ectopy will suppress PESP therapy as, for example, will sinus tachycardia, other SVTs, VTs, and VF. The inventors have discovered that this rule is a key component of safe and effective PESP stimulation therapy in a variety of situations.

The application of PESP therapy according to the embodiments of the invention may be altered by (i) a physician (based on laboratory results and the patient's signs and symptoms), (ii) by the patient (to help with anticipated or present symptoms such as associated with exertion), or (iii) automatically by device sensors that detect conditions responsive to these stimulation therapies. In each of these cases there may be distinct maximal therapy durations and termination criteria (or therapy may be ended by the physician or patient).

Automated sensor-governed initiation of stimulation therapies are described herein. If there is no current arrhythmia, physiologic sensors may be employed to determine if cardiac hemodynamic dysfunction therapy is to be initiated. Blood pressure signals such as arterial, right ventricular, and/or left ventricular pressure sensors (which may be utilized to derive other discrete cardiovascular pressure measurements) may be used to obtain respective pressure measurements. Therapy may be initiated when these measurements indicate a pressure change that drops below or exceeds a predetermined threshold for an established period of time. In one example depicted in detail herein, a severe level of dysfunction (LV dP/dt max<400 mmHg/s) is observed during normal sinus rhythm for over six seconds. The pressure measurements may be weighted and/or combined to obtain a statistic used to trigger therapy delivery. The statistic may be used to develop long-term trend data used to indicate the onset and severity of HF and hemodynamic dysfunction as well as monitor effectiveness of therapy.

In another aspect of these embodiments of the invention, RV pressure is used to derive RV end-diastolic and developed pressure, maximum pressure change as a function of time (dP/dtₘₐₓ), an estimate of pulmonary artery diastolic pressure (ePAD), an RV relaxation or contraction time constant (tau), or RV recirculation fraction (RF). These derived parameters are then used to determine when the degree of dysfunction has exceeded an acceptable level such that therapy delivery is initiated. Parameters could be measured or computed as above and compared to thresholds, or sensor signals could be processed and cardiac dysfunction identified through template matching and classification. Thresholds and/or classification schemes may be periodically updated to reject any natural changes in the condition of the patient as cause for therapy.

The embodiments of the invention may also incorporate predicted hemodynamic compromise through an extended analysis of cardiac cycle-length. For example, a long duration and rapid SVT, VT, or VF has a high likelihood of producing dysfunction including acute HF decompensation, cardiogenic shock, or even electromechanical dissociation (EMD) or pulseless electrical activity (PEA) after spontaneous termination or cardioversion. In such cases, a trial of stimulation therapy might be programmed without mechanical, metabolic, or chemical sensor confirmation.

Other signals such as surface electrocardiogram (ECG) or electrogram (EGM) signals from electrodes within the patient's body may be used to detect dysfunction and heart failure (HF). For example, the ST segment level of a cardiac cycle (PQRST) detected by an ECG may be monitored. An elevated or depressed ST segment level has been found to be reliable indicator of ischemia, a condition known to be associated with dysfunction and HF. Alternatively, the duration of the Q-T interval may also be used to detect hemodynamic dysfunction. For example, a shortened Q-T interval may indicate myocardial dysfunction. A template matching algorithm such as a wavelet classification algorithm may be used to identify electrogram signals that are associated with hemodynamic dysfunction.

Chemical sensors may be used to initiate therapy, including sensors that analyze the blood to detect changes in lactate, O₂ saturation, PO₂, PCO₂ and pH. Expired gas may be analyzed for PCO₂ as an indicator of cardiac output during resuscitation procedures. Therapy is then continued until the degree of dysfunction or HF reflected by these variables is less than a predetermined amount for a sufficient period of time.

Physiologic signals may continue to be sensed to determine if a therapy termination condition is met so that therapy may be terminated. The use, however, of a mechanical sensor such as a pressure sensor or an accelerometer to determine whether or not to apply therapy has the drawback in that external treatments of PEA/EMD such as cardiac chest compressions may introduce error into the physiologic signals, inhibiting or delaying therapy when it may be needed. An additional aspect of these embodiments of the invention is to include not only a mechanical sensor in or on the heart to detect cardiac function, but a second sensor or a multitude of sensors away from the heart, such as inside the implantable device housing or can (acting as an indifferent electrode). From this second sensor, CPR artifact (due to chest compressions and the like) could be identified and, for example, subtracted to reveal a more accurate assessment of true cardiac function.

Therapy is ordinarily automatically interrupted on detection of an arrhythmic event. Upon termination of the arrhythmic event, the therapy may be automatically reconfigured to reduce risk of re-induction. Therapy could also be interrupted on detection of a sufficient quantity of abnormal depolarizations such as premature ventricular contractions (PVC). One or more PVCs could be detected through the use of rate limits or through a template matching type algorithm such as a wavelet classification algorithm, or using a PR-logic® type rhythm discrimination scheme which is a proprietary detection technique of Medtronic, Inc.

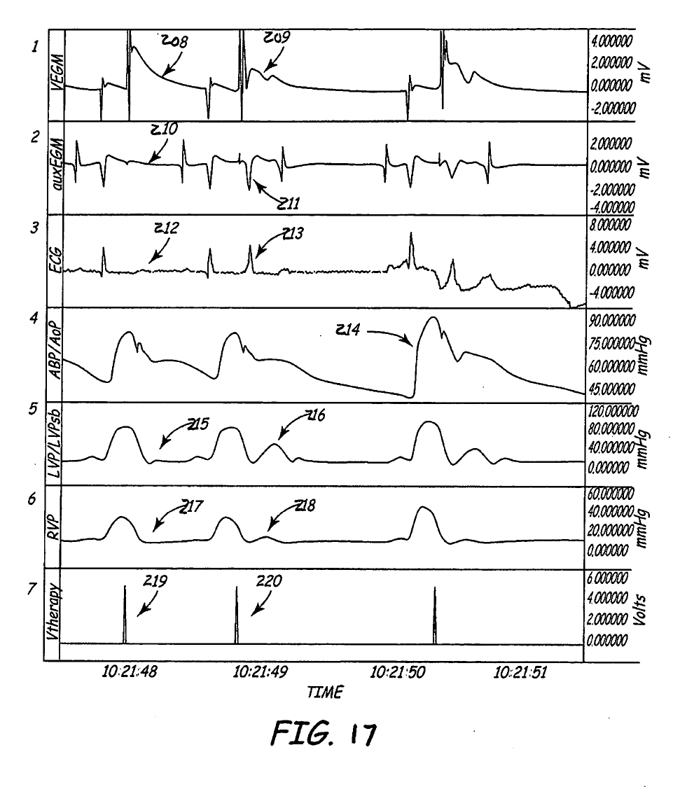

Although beneficial for cardiac function, the delivery of PESP stimulation pulses must be controlled so as to minimize the risk of inducing an arrhythmia. This is best realized with reference to the traces of an ECG or EGM signal aligned with a stimulus-intensity curve (FIG. 16) to show the intensity of pulses required to induce an extrasystole during the time period following a ventricular depolarization which coincides to the QRS complex at an initial time zero (0). During the absolute refractory period, the ventricles are refractory so that another depolarization will not be induced by delivery of electrical stimulation. Following this time, the tissue recovers so that another electrical depolarization is possible upon the delivery of electrical stimulation to the cardiac tissue. The amount of electrical current required to cause the extrasystole during this time is represented by the stimulus-intensity curve.

Initially, after the refractory period, the electrical current level required to capture the tissue is high but thereafter sharply decreases to a baseline level of roughly 0.5-1 mA for an implanted pacing lead.

Also, the “vulnerable period” of the ventricles must be considered when administering PESP therapy. The vulnerable period represents a time period during which an electrical pulse delivered at, or above, a pre-determined amplitude has the risk of inducing a VT or VF episode. For example, a pulse delivered at about 170 ms having an amplitude of 40 mA or more may induce an tachyarrhythmia.

The level of enhancement or potentiation resulting from excitatory PESP stimulation therapy follows a potentiation response curve as further described herein. The inventors have found that such electrical stimulation pulses delivered shortly after the refractory period ends produce strong subsequent contractions. Further delays of the stimulation diminish the amount of potentiation. Stimulation too early (i.e., prematurely) results in no additional potentiation at all since the myocardium is refractory. As discussed with respect to the vulnerable period, the risk of arrhythmia induction is confined to a relatively narrow time interval just slightly longer than the refractory period. However, the inventors have discovered that such a risk is quite low if single low amplitude PESP pulses are delivered according to delivery lockout rules (such as briefly described above). The low amplitude PESP pulse is essentially “benefit neutral” when restricted to the absolute refractory period, is not without risk for a short period just slightly longer then the refractory period, rises to a maximum benefit shortly after this short period, and finally declines to again become approximately “benefit neutral” for pulses delivered near the intrinsic cycle length.

As a result, it is apparent that stimulation timing with respect to the refractory-nonrefractory period boundary is a critical aspect of obtaining the desired response (PESP) and controlling risks and benefits of therapy delivery. The embodiments of the invention provide for means to determine this time from electrical, and/or mechanical sensor signals and thereby enable safer and more effective stimulation therapies.

The inventors exploit the fact that the refractory period is closely associated with the Q-T interval, which may be derived from electrogram signals or other physiologic sensor signals by techniques known in the art. The Q-T interval length is used to estimate the duration of the refractory period either directly, or by incorporating a function of heart rate and sensing delays. In the case of PESP therapy, the Q-T interval length can be estimated by the time interval from an extrasystole stimulation pulse to an evoked T wave and would be slightly longer than during a cardiac cycle not associated with PESP. This is because the extra depolarization caused by the PESP prolongs the QT interval slightly.

Alternatively, an evoked response of the PESP stimulation could be monitored to indicate whether the PESP therapy was delivered in the refractory period or not. For example, a number of electrical pulses are applied to the myocardium, beginning during the refractory period. The result of each pulse is sensed on an EGM from either the stimulating electrode or an auxiliary electrode until an evoked response is sensed, indicating that the pulse caused an extrasystole. At this point, no further pulses would be applied to minimize the risk of inducing arrhythmias.

In another example, a single pulse's amplitude and timing may be manipulated until capture is detected by an evoked R wave. If capture is lost, the stimulus pulse is delayed more, or amplitude increased, or the number of pulses in a PESP pulse train is increased. Also, the characteristics of a pressure waveform (or any other mechanical response variable) used to assess whether the PESP stimulation is/was capturing the ventricles can be utilized when practicing the embodiments of the invention. The presence of the extrasystole could be identified by a small ventricular pressure pulse 5-80% of the size of the preceding pressure pulse or through a suitable algorithm such as a template-matching algorithm. A transition between capture and noncapture for a pulse intended to serve as an extrasystole may also be identified by a change in the pressure waveform of the subsequent potentiated beat. This can be clearly illustrated with respect to the arterial pulse pressure.

As the refractory-nonrefractory boundary is very important and varies from patient to patient, ventricular site to ventricular site, and even with a patient over time, with disease and drugs, these methods are to be employed periodically or continually to the stimulation timing algorithm portion of the device. If this boundary information is not used to set pulse timing directly, it may be employed to establish limits for the timing that are in turn set by a clinician or some automatic control algorithm such as that described next.

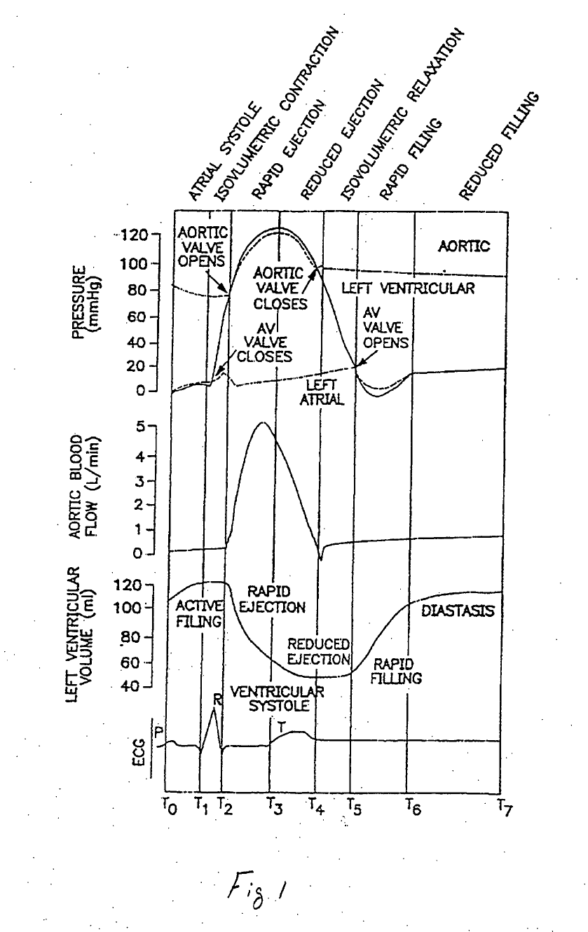

A representative heart and cardiovascular system is influenced by electrical therapies including pacing, defibrillation, CRT, and PESP stimulation therapy. The heart and cardiovascular system may be monitored by electrical, mechanical, and metabolic/chemical sensors. The signals from these sensors influence decisions to start or stop therapy, closed loop control, refractory period detection, therapy delivery lockout rules, and atrial coordinated pacing. Before describing embodiments of the invention, reference is made to FIG. 1 which cardiac cycle in relation to the fluctuations in absolute blood pressure, aortic blood flow and ventricular volume in the left heart. The right atria and ventricles exhibit roughly similar pressure, flow, and volume fluctuations, in relation to the PQRST complex, as the left atria and ventricles. It is understood that the monitoring and stimulation therapy aspects of these embodiments of the invention may reside and act on either or both sides of the heart. The cardiac cycle is completed in the interval between successive PQRST complexes and following relaxation of the atria and ventricles as the right and left atria re-fill with venous blood and oxygenated blood. In sinus rhythm, the interval between depolarizations may be on the order of 500 ms to 1,000 ms for a corresponding sinus heart rate of 120 bpm to 60 bpm, respectively. In this time interval, the atria and ventricles are relaxed, and overall atrial size or volume may vary as a function of pleural pressure and respiration. In the blood pressure diagrams of FIG. 1, it may be observed that the atrial and ventricular blood pressure changes track and lag the P-waves and R-waves of the cardiac cycle. The time period T₀-T₁ encompasses the AV interval.

In patients suffering from cardiac insufficiency arising from bradycardia due to an incompetent SA node or AV-block, atrial and/or ventricular conventional pacing may be prescribed to restore a sufficient heart rate and AV synchrony. In FIG. 1, for example, atrial and/or ventricular pacing pulses would precede the P-wave and the deflection of the QRS complex commonly referred to as the R-wave. Cardiac output may be reduced by the inability of the atrial or ventricular myocardial cells to relax following atrial (T₀-T₁) and ventricular (T₁-T₄) systolic periods. Prolonged systolic time periods reduce passive filling time T₄-T₇ as shown in FIG. 1. Thus, the amount of blood expelled from the atria and/or ventricles in the next cardiac cycle may be less than optimum. This is particularly the case with CHF (Congestive Heart Failure) patients or other patients in whom the stiffness of the heart is increased, cardiac filling during the passive filling phase (T₄-T₇) and during atrial systole (T₀-T₁) can be significantly limited.

It will be appreciated from the following description that the implantable medical device (IMD) of the embodiments of the invention may be utilized to obtain the aforementioned parameters as stored patient data over a period of time and to deliver therapies for treating the heart failure. The IMD can then determine whether a particular therapy is appropriate. While the embodiments of the invention are described with respect to PESP stimulation, other therapies delivered can include drug therapies and electrical stimulation therapies, and pacing therapies including single chamber, dual chamber and multi-chamber (bi-atrial and/or bi-ventricular) pacing.

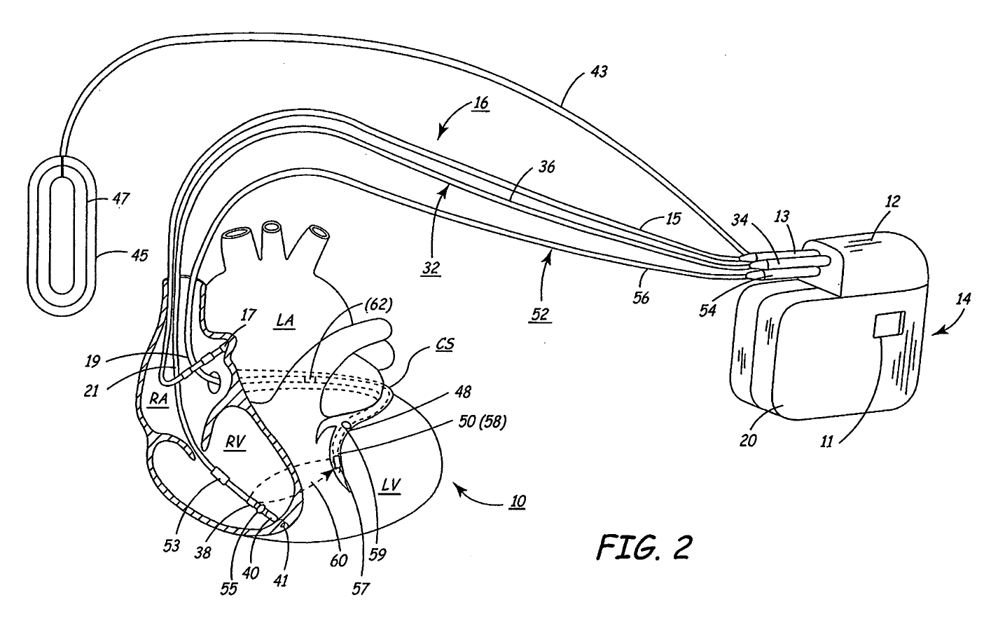

In FIG. 2, heart 10 includes the upper heart chambers, the right atrium (RA) and left atrium (LA), and the lower heart chambers, the right ventricle (RV) and left ventricle (LV) and the coronary sinus (CS) extending from the opening in the right atrium laterally around the atria to form the great vein that extends further inferiorly into branches of the great vein. The cardiac cycle commences normally with the generation of the depolarization impulse at the SA Node in the right atrial wall. The impulse then conducts through the right atrium by way of internodal tracts, and conducts to the left atrial septum by way of Bachmann's bundle. The RA depolarization wave reaches the atrio-ventricular (AV) node and the atrial septum within about 40 msec and reaches the furthest walls of the RA and LA within about 70 msec. Approximately 50 ms following electrical activation, the atria contract. The aggregate RA and LA depolarization wave appears as the P-wave of the PQRST complex when sensed across external ECG electrodes and displayed. The component of the atrial depolarization wave passing in proximity to a unipolar or pair of bipolar pace/sense electrodes, respectively, located on or adjacent the RA or LA is also referred to as a sensed P-wave. Although the location and spacing of the external ECG electrodes or implanted unipolar atrial pace/sense electrodes has some influence, the normal P-wave width does not exceed 80 msec in width as measured by a high impedance sense amplifier coupled with such electrodes. A normal near field P-wave sensed between closely spaced bipolar pace/sense electrodes and located in or adjacent the RA or the LA has a width of no more than 60 msec as measured by a high impedance sense amplifier.

The depolarization impulse that reaches the AV Node conducts down the bundle of His in the intraventricular septum after a delay of about 120 msec. The depolarization wave reaches the apical region of the heart about 20 msec later and is then travels superiorly though the Purkinje Fiber network over the remaining 40 msec. The aggregate RV and LV depolarization wave and the subsequent T-wave accompanying re-polarization of the depolarized myocardium are referred to as the QRST portion of the PQRST cardiac cycle complex when sensed across external ECG electrodes and displayed. When the amplitude of the QRS ventricular depolarization wave passing by a bipolar or unipolar pace/sense electrode pair located on or adjacent to the myocardium exceeds a threshold amplitude, it is detected as a sensed R-wave. Although the location and spacing of the external ECG electrodes or implanted unipolar ventricular pace/sense electrodes has some influence on R-wave sensing, the normal R-wave duration does not exceed 80 msec as measured by a high impedance sense amplifier. A normal near field R-wave sensed between closely spaced bipolar pace/sense electrodes and located in or adjacent the RV or the LV has a width of no more than 60 msec as measured by a high impedance sense amplifier.

The normal electrical activation sequence can become highly disrupted in patients suffering from advanced HF and can manifest itself as an intra-atrial conduction delay (IACD), left bundle branch block (LBBB), right bundle branch block (RBBB), and/or intraventricular conduction delay (IVCD). These conduction defects give rise to dyssynchrony between RV and LV activation as well as intra-ventricular dyssynchrony. In RBBB and LBBB patients, the QRS complex is widened beyond the normal range to between 120 msec and 250 msec as measured on surface ECG. This increased width demonstrates the lack of synchrony of the right and left ventricular depolarizations which is often linked to dysynchronous contraction.

FIG. 2 also depicts an implanted, multi-channel cardiac pacemaker, ICD, IPG (implantable pulse generator) or other IMD of the above noted types for restoring AV synchronous contractions of the atrial and ventricular chambers and simultaneous or sequential pacing of the right and left ventricles. The pacemaker IPG 14 is implanted subcutaneously in a patient's body between the skin and the ribs. Three endocardial leads 16, 32, and 52 connect the IPG 14 with the RA, the RV and the LV, respectively. Each lead has at least one electrical conductor and pace/sense electrode, and a remote indifferent can electrode 20 is formed as part of the outer surface of the housing of the IPG 14. As described further below, the pace/sense electrodes and the remote indifferent can electrode 20 (IND_CAN electrode) can be selectively employed to provide a number of unipolar and bipolar pace/sense electrode combinations for pacing and sensing functions. The depicted positions in or about the right and left heart chambers are also merely exemplary. Moreover other leads and pace/sense electrodes may be used instead of the depicted leads and pace/sense electrodes that are adapted to be placed at electrode sites on or in or relative to the RA, LA, RV and LV.

The depicted bipolar endocardial RA lead 16 is passed through a vein into the RA chamber of the heart 10, and the distal end of the RA lead 16 is attached to the RA wall by an attachment mechanism 17. The bipolar endocardial RA lead 16 is formed with an in-line connector 13 fitting into a bipolar bore of IPG connector block 12 that is coupled to a pair of electrically insulated conductors within lead body 15 and connected with distal tip RA pace/sense electrode 19 and proximal ring RA pace/sense electrode 21. Delivery of atrial pace pulses and sensing of atrial sense events is effected between the distal tip RA pace/sense electrode 19 and proximal ring RA pace/sense electrode 21, wherein the proximal ring RA pace/sense electrode 21 functions as an indifferent electrode (IND_RA). Alternatively, a unipolar endocardial RA lead could be substituted for the depicted bipolar endocardial RA lead 16 and be employed with the IND_CAN electrode 20. Or, one of the distal tip RA pace/sense electrode 19 and proximal ring RA pace/sense electrode 21 can be employed with the IND_CAN electrode 20 for unipolar pacing and/or sensing.

Bipolar, endocardial RV lead 32 is passed through the vein and the RA chamber of the heart 10 and into the RV where its distal ring and tip RV pace/sense electrodes 38 and 40 are fixed in place in the apex by a conventional distal attachment mechanism 41. The RV lead 32 is formed with an in-line connector 34 fitting into a bipolar bore of IPG connector block 12 that is coupled to a pair of electrically insulated conductors within lead body 36 and connected with distal tip RV pace/sense electrode 40 and proximal ring RV pace/sense electrode 38, wherein the proximal ring RV pace/sense electrode 38 functions as an indifferent electrode (IND_RV). Alternatively, a unipolar endocardial RV lead could be substituted for the depicted bipolar endocardial RV lead 32 and be employed with the IND_CAN electrode 20. Or, one of the distal tip RV pace/sense electrode 40 and proximal ring RV pace/sense electrode 38 can be employed with the IND_CAN electrode 20 for unipolar pacing and/or sensing.

In this illustrated embodiment, a unipolar, endocardial LV CS lead 52 is passed through a vein and the RA chamber of the heart 10, into the CS and then inferiority in a branching vessel of the great vein 48 to extend the distal LV CS pace/sense electrode 50 alongside the LV chamber. The distal end of such LV CS leads is advanced through the superior vena cava, the right atrium, the ostium of the coronary sinus, the coronary sinus, and into a coronary vein descending from the coronary sinus, such as the great vein. Typically, LV CS leads and LA CS leads do not employ any fixation mechanism and instead rely on the close confinement within these vessels to maintain the pace/sense electrode or electrodes at a desired site. The LV CS lead 52 is formed with a small diameter single conductor lead body 56 coupled at the proximal end connector 54 fitting into a bore of IPG connector block 12. A small diameter unipolar lead body 56 is selected in order to lodge the distal LV CS pace/sense electrode 50 deeply in a vein branching inferiority from the great vein 48.

Preferably, the distal, LV CS active pace/sense electrode 50 is paired with the proximal RV defibrillator coil 53 or can 20 for delivering LV pace pulses. The distal LV CS active pace/sense electrode 50 is also preferably paired with the distal tip RV active pace/sense electrode 40 for sensing across the RV and LV as described further below.

Moreover, in a four-chamber embodiment, LV CS lead 52 could additionally bear a proximal LA CS pace/sense electrode positioned along the lead body to lie in the larger diameter coronary sinus CS adjacent the LA. In that case, the lead body 56 would encase two electrically insulated lead conductors extending proximally from the more proximal LA CS pace/sense electrode(s) and terminating in a bipolar connector 54. The LV CS lead body may also be smaller between the proximal LA CS electrode and the distal LV CS active pace/sense electrode 50. RA pacing and sensing could occur between electrode 17 and housing 20.

Typically, in pacing/defibrillation systems of the type illustrated in FIG. 2, the electrodes designated above as “pace/sense” electrodes are used for both pacing and sensing functions. In accordance with one aspect of the embodiments of the invention, these “pace/sense” electrodes can be selected to be used exclusively as pace or sense electrodes or to be used in common as pace/sense electrodes in programmed combinations for sensing cardiac signals and delivering pace pulses along pacing and sensing vectors. Separate or shared indifferent pace and sense electrodes can also be designated in pacing and sensing functions. For convenience, the following description separately designates pace and sense electrode pairs where a distinction is appropriate. With respect to the embodiments of the invention, a subcutaneous electrode 45 coupled to medical electrical lead 43 may be added to or substituted for one or more of the leads or electrodes depicted in FIG. 2. If a subcutaneous electrode 45 is utilized, a suitable defibrillation coil 47 may be coupled to appropriate high voltage circuitry to deliver a timed defibrillation pulse. While coil electrode 53 is depicted coupled to a portion of RV lead 32, such an electrode may be coupled to other portions of any of the leads depicted in FIG. 2, such as LV electrode 57. The coil electrode 53, subcutaneous electrode 45 or other types of suitable electrode configurations may be electrically coupled to low voltage pacing/sensing circuitry in addition to high voltage circuitry. As is known, such electrodes may be disposed in a variety of locations in, around and on the heart.

Also depicted in FIG. 2 is an RV sensor 55 and an LV sensor 59 which may comprise one or more of a variety of sensors as is known in the art. Preferably RV sensor 55 comprises an absolute pressure sensor, but other pressure sensors may be utilized. In addition, RV sensor 55 may comprise an accelerometer, an impedance electrode, a saturated oxygen sensor, a pH sensor, and the like. In addition, each of the leads could carry a mechanical sensor for developing systolic and diastolic pressures and a series of spaced apart impedance sensing leads for developing volumetric measurements of the expansion and contraction of the RA, LA, RV and LV.

Of course, such sensors must be rendered biocompatible and reliable for long-term use. In addition, one or more sensors may be disposed in or on the housing 20 of IMD 14 such as sensor 11 depicted in FIG. 2.

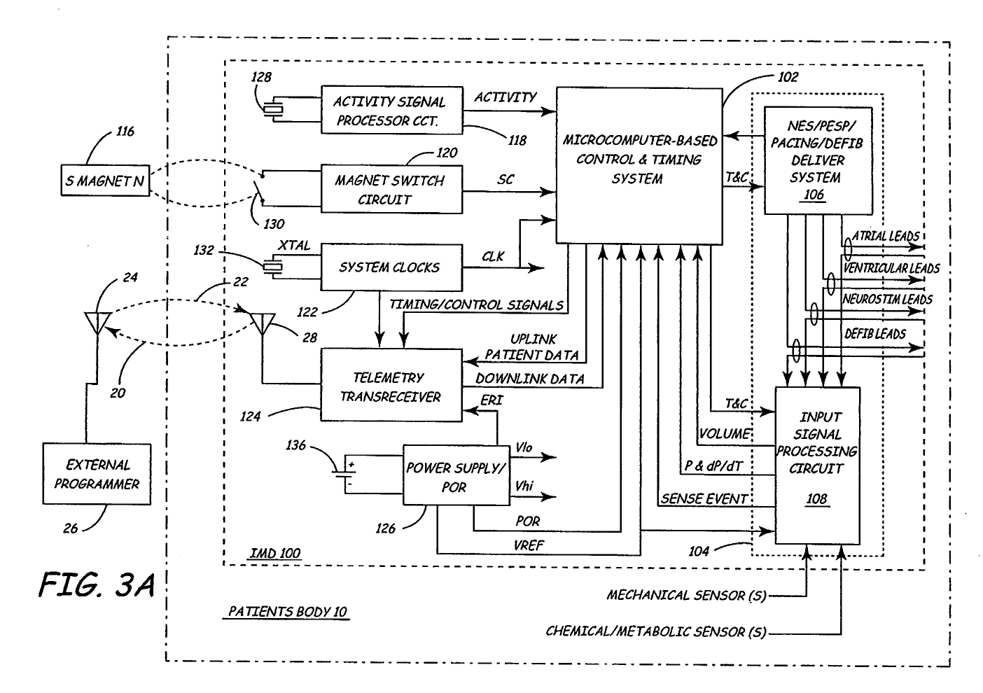

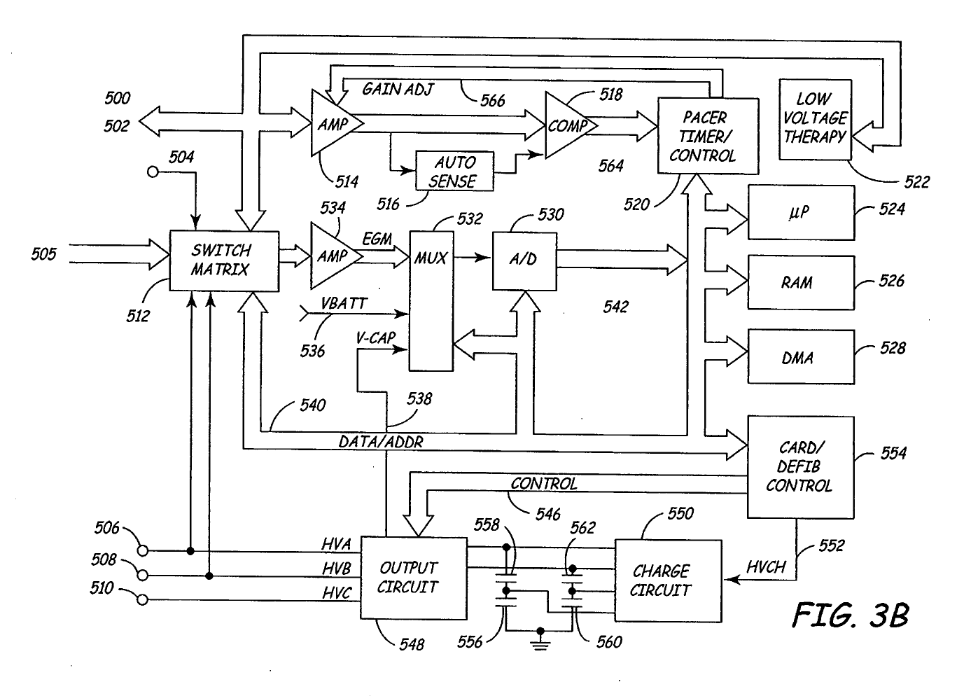

FIG. 3A depicts a system architecture of an exemplary multi-chamber IMD 100 implanted into a patient's body 10 that provides delivery of a therapy and/or physiologic input signal processing. The typical multi-chamber monitor/sensor 100 has a system architecture that is constructed about a microcomputer-based control and timing system 102 that varies in sophistication and complexity depending upon the type and functional features incorporated therein. The functions of microcomputer-based multi-chamber monitor/sensor control and timing system 102 are controlled by firmware and programmed software algorithms stored in RAM and ROM including PROM and EEPROM and are carried out using a CPU, ALU, etc., of a typical microprocessor core architecture. Of course, such firmware and software may be modified in situ (e.g., in vivo) and the operational characteristics may be adapted for a particular situation or patient. A physician or clinician may change one or more parameters which will cause a change in the detection or response of such algorithms. Oftentimes, discrete values may be changed such that a desired software routine is advantageously altered, although sometimes an entirely new set of operating software may be substituted for an existing set of operating software, as is known in the art. The microcomputer-based multi-chamber monitor/sensor control and timing system 102 may also include a watchdog circuit, a DMA controller, a block mover/reader, a CRC calculator, and other specific logic circuitry coupled together by on-chip data bus, address bus, power, clock, and control signal lines in paths or trees in a manner well known in the art. It will also be understood that control and timing of multi-chamber monitor/sensor 100 can be accomplished with dedicated circuit hardware or state machine logic rather than a programmed micro-computer.

The multi-chamber monitor/sensor 100 also typically includes patient interface circuitry 104 for receiving signals from sensors and pace/sense electrodes located at specific sites of the patient's heart chambers and/or delivering PESP stimulation to derive heart failure parameters or a pacing therapy to the heart chambers. The patient interface circuitry 104 therefore comprises a PESP stimulation delivery system 106 optionally including pacing and other stimulation therapies and a physiologic input signal processing circuit 108 for processing the blood pressure and volumetric signals output by sensors. For purposes of illustration of the possible uses of these embodiments of the invention, a set of lead connections are depicted for making electrical connections between the therapy delivery system 106 and the input signal processing circuit 108 and sets of pace/sense electrodes located in operative relation to the RA, LA, RV and LV.

As depicted in FIG. 3A, chemical/metabolic sensor input and/or mechanical sensor inputs are provided to the input signal processing circuit 108. As described with respect to FIG. 2, a wide variety of such sensors may be utilized when practicing the embodiments of the invention.

A battery provides a source of electrical energy to power the multi-chamber monitor/sensor operating system including the circuitry of multi-chamber monitor/sensor 100 and to power any electromechanical devices, e.g., valves, pumps, etc. of a substance delivery multi-chamber monitor/sensor, or to provide electrical stimulation energy of an ICD shock generator, cardiac pacing pulse generator, or other electrical stimulation generator. The typical energy source is a high energy density, low voltage battery 136 coupled with a power supply/POR circuit 126 having power-on-reset (POR) capability. The power supply/POR circuit 126 provides one or more low voltage power Vlo, the POR signal, one or more VREF sources, current sources, an elective replacement indicator (ERI) signal, and, in the case of an ICD, high voltage power Vhi to the therapy delivery system 106.

Virtually all current electronic multi-chamber monitor/sensor circuitry employs clocked CMOS digital logic ICs that require a clock signal CLK provided by a piezoelectric crystal 132 and system clock 122 coupled thereto as well as discrete components, e.g., inductors, capacitors, transformers, high voltage protection diodes, and the like that are mounted with the ICs to one or more substrate or printed circuit board. In FIG. 3A, each CLK signal generated by system clock 122 is routed to all applicable clocked logic via a clock tree. The system clock 122 provides one or more fixed frequency CLK signal that is independent of the battery voltage over an operating battery voltage range for system timing and control functions and in formatting uplink telemetry signal transmissions in the telemetry I/O circuit 124.

The RAM registers may be used for storing data compiled from sensed cardiac activity and/or relating to device operating history or sensed physiologic parameters for uplink telemetry transmission on receipt of a retrieval or interrogation instruction via a downlink telemetry transmission. The criteria for triggering data storage can also be programmed in via downlink telemetry transmitted instructions and parameter values The data storage is either triggered on a periodic basis or by detection logic within the physiologic input signal processing circuit 108 upon satisfaction of certain programmed-in event detection criteria. In some cases, the multi-chamber monitor/sensor 100 includes a magnetic field sensitive switch 130 that closes in response to a magnetic field, and the closure causes a magnetic switch circuit to issue a switch closed (SC) signal to control and timing system 102 which responds in a magnet mode. For example, the patient may be provided with a magnet 116 that can be applied over the subcutaneously implanted multi-chamber monitor/sensor 100 to close switch 130 and prompt the control and timing system to deliver a therapy and/or store physiologic episode data when the patient experiences certain symptoms. In either case, event related data, e.g., the date and time, may be stored along with the stored periodically collected or patient initiated physiologic data for uplink telemetry in a later interrogation session.

In the multi-chamber monitor/sensor 100, uplink and downlink telemetry capabilities are provided to enable communication with either a remotely located external medical device or a more proximal medical device on the patient's body or another multi-chamber monitor/sensor in the patient's body as described above with respect to FIG. 2 and FIG. 3A (and FIG. 3B described below). The stored physiologic data of the types described above as well as real-time generated physiologic data and non-physiologic data can be transmitted by uplink RF telemetry from the multi-chamber monitor/sensor 100 to the external programmer or other remote medical device 26 in response to a downlink telemetered interrogation command. The real-time physiologic data typically includes sampled signal waveforms (e.g. intracardiac EGM or pressure waveforms), waveform derived parameters (e.g. dP/dtₘₐₓ or intracardiac electrocardiogram amplitude values), and sensor output signals. The non-physiologic patient data includes currently programmed device operating modes and parameter values, battery condition, device ID, patient ID, implantation dates, device programming history, real time event markers, and the like. In the context of implantable pacemakers and ICDs, such patient data includes programmed sense amplifier sensitivity, pacing or cardioversion pulse amplitude, energy, and pulse width, pacing or cardioversion lead impedance, and accumulated statistics related to device performance, e.g., data related to detected arrhythmia episodes and applied therapies. The multi-chamber monitor/sensor thus develops a variety of such real-time or stored, physiologic or non-physiologic, data, and such developed data is collectively referred to herein as “patient data.”

The physiologic input signal processing circuit 108 therefore includes at least one electrical signal amplifier circuit for amplifying, processing and in some cases detecting sense events from characteristics of the electrical sense signal or sensor output signal. The physiologic input signal processing circuit 108 in multi-chamber monitor/sensors providing dual chamber or multi-site or multi-chamber monitoring and/or pacing functions includes a plurality of cardiac signal sense channels for sensing and processing cardiac signals from sense electrodes located in relation to a heart chamber. Each such channel typically includes a sense amplifier circuit for detecting specific cardiac events and an EGM amplifier circuit for providing an EGM signal to the control and timing system 102 for sampling, digitizing and storing or transmitting in an uplink transmission. Atrial and ventricular sense amplifiers include signal processing stages for detecting the occurrence of a P-wave or R-wave, respectively and providing an ASENSE or VSENSE event signal to the control and timing system 102. Timing and control system 102 responds in accordance with its particular operating system to deliver or modify a pacing therapy, if appropriate, or to accumulate data for uplink telemetry transmission or to provide a Marker Channel® signal in a variety of ways known in the art.

In addition, the input signal processing circuit 108 includes at least one physiologic sensor signal processing channel for sensing and processing a sensor derived signal from a physiologic sensor located in relation to a heart chamber or elsewhere in the body.

Now turning to FIG. 3B, another system architecture for use in conjunction with the embodiments of the invention is depicted. FIG. 3B is an exemplary system that may be utilized to deliver therapy by incorporating the system and method described above. Notably, the depicted system includes a sense amplifier 534 to sense electrical signals such as EGM signals using one or more leads placed within a respective chamber of the heart. These signals are used to determine atrial and ventricular depolarizations and Q-T length so that PESP delivery is provided in an optimized manner. One or more physiological or hemodynamic signals may be sensed using sensors such as those discussed above. These additional signals, which are shown collectively provided on line 505, may be used to determine cardiac output so that therapy may be initiated, terminated, and/or optimized.

The system of FIG. 3B further includes a timer/controller to control the delivery of pacing pulses on output lines 500 and 502. This circuit, alone or in conjunction with microprocessor 524, controls interval lengths, pulse amplitudes, pulse lengths, and other waveform attributes associated with the PESP pulses. Output circuit 548 delivers high-voltage stimulation such as defibrillation shocks under the control of defibrillation control circuit 554.

Not all of the conventional interconnections of these voltages and signals are shown in either FIG. 3A or FIG. 3B and many other variations on the illustrated electronic circuitry are possible, as is known to those of skill in the art.

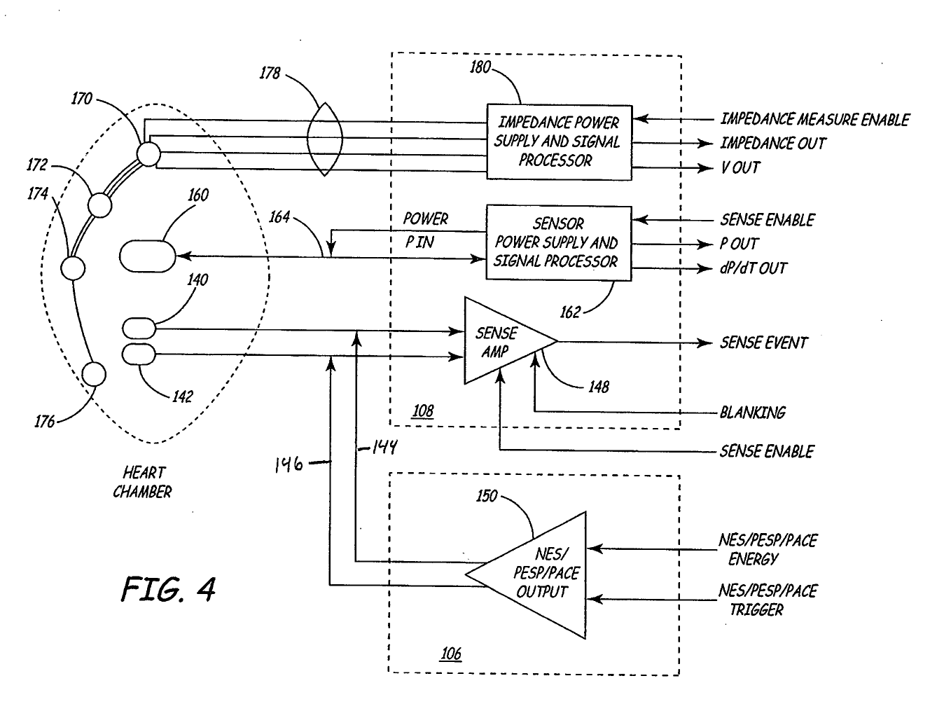

FIG. 4 schematically illustrates one pacing, sensing, and parameter measuring channel in relation to one heart chamber. A pair of pace/sense electrodes 140,142, a sensor 160 (e.g., a pressure, saturated oxygen, flow, pH or the like), and a plurality, e.g., four, impedance measuring electrodes 170,172,174,176 are located in operative relation to the heart chamber. The pair of pace/sense electrodes 140, 142 are located in operative relation to the heart chamber and coupled through lead conductors 144 and 146, respectively, to the inputs of a sense amplifier 148 located within the input signal processing circuit 108. The sense amplifier 148 is selectively enabled by the presence of a sense enable signal that is provided by control and timing system 102. The sense amplifier 148 is enabled during prescribed times when pacing is either enabled or not enabled as described below in reference to the measurement of the parameters of heart failure. The blanking signal is provided by control and timing system 102 upon delivery of a pacing or PESP pulse or pulse train to disconnect the sense amplifier inputs from the lead conductors 144 and 146 for a short blanking period in a manner well known in the art. When sense amplifier 148 is enabled and is not blanked, it senses the electrical signals of the heart, referred to as the EGM, in the heart chamber. The sense amplifier provides a sense event signal signifying the contraction of the heart chamber commencing a heart cycle based upon characteristics of the EGM, typically the P-wave when the heart chamber is the RA or LA and the R-wave, when the heart chamber is the RV or LV, in a manner well known in the pacing art. The control and timing system responds to non-refractory sense events by restarting an escape interval (EI) timer timing out the EI for the heart chamber, in a manner well known in the pacing art.

The pair of pace/sense electrodes 140, 142 are also coupled through lead conductors 144 and 146, respectively, to the output of a pulse generator 150. The pulse generator 150, within PESP/pacing delivery system 106, selectively provides a pacing pulse to electrodes 140, 142 in response to a PESP/PACE trigger signal generated at the time-out of the EI timer within control and timing system 102 in a manner well known in the pacing art. Or, the pulse generator 150 selectively provides a PESP pulse or pulse train to electrodes 140, 142 in response to a PESP/PACE trigger signal generated at the time-out of an ESI timer within control and timing system 102 in the manner described in the above-referenced '098 patent to cause the heart chamber to contract more forcefully, the increased force depending upon the duration of the ESI.

The sensor 160 and/or other physiologic sensor is coupled to a sensor power supply and signal processor 162 within the input signal processing circuit 108 through a set of lead conductors 164 that convey power to the sensor 160 and sampled blood pressure P signals from the sensor 160 to the sensor power supply and signal processor 162. The sensor power supply and signal processor 162 samples the blood pressure impinging upon a transducer surface of the sensor 160 located within the heart chamber when enabled by a sense enable signal from the control and timing system 102. As an example, absolute pressure P, developed pressure DP and pressure rate of change dP/dt sample values can be developed by sensor power supply and signal processor unit 162 or by the control and timing system 102 for storage and processing as described further below. The sensor 160 and a sensor power supply and signal processor 162 may take the form disclosed in commonly assigned U.S. Pat. No. 5,564,434.

The set of impedance electrodes 170, 172, 174 and 176 is coupled by a set of conductors 178 and is formed as a lead of the type described in the above-referenced '717 patent that is coupled to the impedance power supply and signal processor 180. Impedance-based measurements of cardiac parameters such as stroke volume are known in the art. The spaced apart electrodes can also be disposed along impedance leads lodged in cardiac vessels, e.g., the coronary sinus and great vein or attached to the epicardium around the heart chamber. The impedance lead may be combined with the pace/sense and/or pressure sensor bearing lead.

A measure of heart chamber volume V is provided by the set of impedance electrodes 170, 172, 174 and 176 when the impedance power supply and signal processor 180 is enabled by an impedance measure enable signal provided by control and timing system 102. A fixed current carrier signal is applied between the pairs of impedance electrodes and the voltage of the signal is modulated by the impedance through the blood and heart muscle which varies as distance between the impedance electrodes varies. Thus, the calculation of the heart chamber volume V signals from impedance measurements between selected pairs of impedance electrodes 170, 172, 174 and 176 occurs during the contraction and relaxation of the heart chamber that moves the spaced apart electrode pairs closer together and farther apart, respectively, due to the heart wall movement or the tidal flow of blood out of and then into the heart chamber. Raw signals are demodulated, digitized, and processed to obtain an extrapolated impedance value. When this value is divided into the product of blood resistivity times the square of the distance between the pairs of spaced electrodes, the result is a measure of instantaneous heart chamber volume V within the heart chamber.

In accordance with the embodiments of the invention, the IMD measures a group of parameters indicative of the state of heart failure employing EGM signals, measures of absolute blood pressure P and/or dP/dt, saturated oxygen, flow, pH or the like and measures of heart chamber volume V over one or more cardiac cycles.

The steps of deriving the RF, MR, E_{ES}, and tau parameters indicative of the state of heart failure are more fully described in U.S. Pat. No. 6,738,667 and will not be repeated here. For the uninitiated the following description is provided; however, if additional details are desired the reader is directed to the '667 disclosure. These parameters are determined periodically throughout each day regardless of patient posture and activity. However, the patient may be advised by the physician to undertake certain activities or movements at precise times of day or to simultaneously initiate the determination of the parameters though use of a magnet or a remote system programmer unit (not depicted) that is detected by the IMD. Certain of the parameters are only measured or certain of the parameter data are only stored when the patient heart rate is within a normal sinus range between programmed lower and upper heart rates and the heart rhythm is relatively stable. The parameter data and related data, e.g., heart rate and patient activity level, are date and time stamped and stored in IMD memory for retrieval employing conventional telemetry systems. Incremental changes in the stored data over time provide a measure of the degree of change in the heart failure condition of the heart. Such parameter data and related data may be read, reviewed, analyzed and the like and the parameter data may be changed based on a current patient condition, a patient history, patient or physician preference(s) and the like.

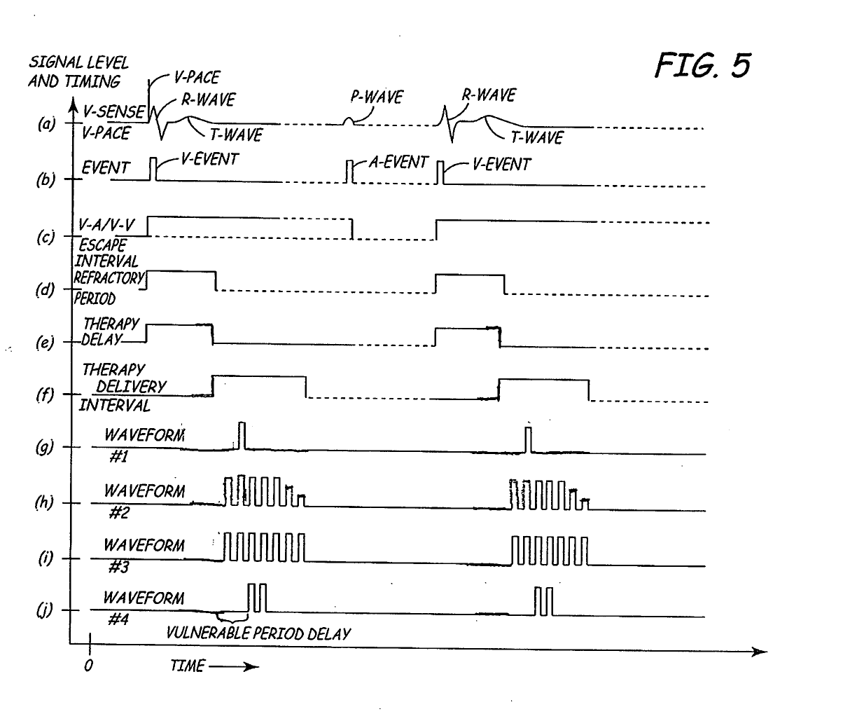

Turning to FIG. 5, the timing diagram illustrates the timing of delivery of stimulation to a heart chamber in relation to a timed interval from a sensed or paced event as well as alternative pulse waveforms of the PESP stimulation. In accordance with one aspect of the embodiments of the invention, a therapeutic stimulation delay illustrated in tracing (e) is timed out from a sensed or paced event (e.g., the illustrated V-EVENTs). A stimulus pulse is delivered to the atria and/or ventricles in the depicted therapy delivery interval of tracing (f) commencing after time-out of the delay. The pulses for PESP therapy delivery are intended to be supra-threshold in nature, that is, of sufficient energy to depolarize the heart when they are delivered in the non-refractory period of the heart cycle so that the heart is captured by at least one of the PESP pulses falling outside the refractory period. Initial pulses delivered during the refractory period can also potentiate the heart. For simplicity of illustration, the tracings (f)-(j) are expanded in length, and the depolarization of the heart that they cause is not depicted in tracing (a). The amplitude and number of refractory interval pulses and PESP pulses in each therapy pulse train and the spacing between the pulses may also differ from the illustrated tracings (g)- (j).

The ventricular sense or pace event detected in tracing (b) also triggers the timing out of an escape interval in tracing (c) which may be terminated by the sensing of a subsequent atrial or ventricular event, depending on the operating mode of the system. The first depicted sequence in FIG. 5 shows the full time-out of the escape interval in tracing (c), the refractory period in tracing (d), and the therapy delay and delivery intervals in tracings (e) and (f). The therapy delay and therapy delivery intervals can be derived as a function of an intrinsic V-V or V-A escape interval derived by measuring and averaging intervals between intrinsic ventricular and/or atrial sense events or paced events. The therapy delay can also be determined from a measurement of the Q-T interval or from previously executed algorithms that probe the edge of the refractory period and look for mechanical or electrical responses. As illustrated, the therapy delay in tracing (e) delays delivery of the therapy pulse train until the QRS complex ends about 40-60 ms after the V-EVENT well before the start of the vulnerable period of the heart which occurs near the end of the T-wave. The therapy delivery interval is timed to time-out well before the end of the previously derived V-V or V-A escape interval, but is extended for ease of illustration of the pulse trains in tracings (f)- (j).

The therapy stimulation energy is delivered in the form of one or more constant or variable energy stimulation pulses separated by a pulse separation interval between each pulse of the burst. All of the pulses can have the same amplitude and energy as shown in waveform 3 of tracing (i). Or the leading and/or trailing pulses of the pulse train can have ramped amplitudes similar to the waveform 2 illustrated in tracings (h). In tracing (h), the ramp up leading edge amplitudes of a sub-set of the pulses of the burst are shown increasing from an initial amplitude to a maximum amplitude and the ramp down trailing edge amplitudes of a further sub-set of the pulses of the burst are shown decreasing from the maximum amplitude to a terminating amplitude.

The therapy delivery capability is preferably implemented into a system that may include conventional pacing therapies and operating modes as well as cardioversion/defibrillation capabilities or as a stand alone system for simply providing pulse therapies to effect potentiation of myocardial cells between sensed PQRST complexes shown in FIG. 5.

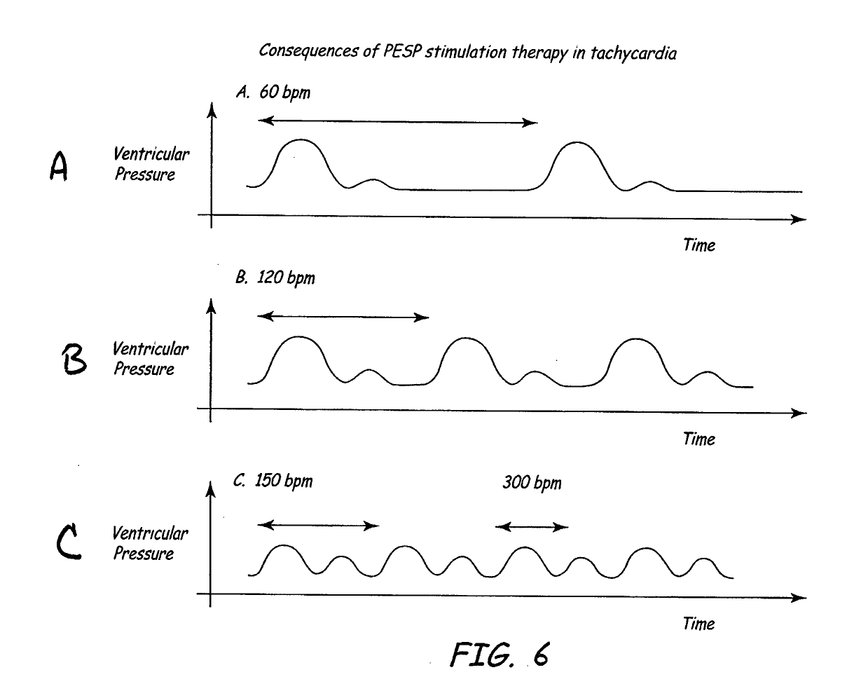

FIG. 6A through 6C illustrate the consequences of PESP stimulation during a tachycardia event. The inventors have discovered that it is preferable, if not absolutely necessary, to cease delivery of excitatory PESP stimulation therapy during tachycardias. In the condition depicted in FIG. 6A, the ventricular mechanical rate is low (60 bpm), the amplitude of the potentiation is large, and there is sufficient time in diastole for ventricular filling. In the condition depicted in FIG. 6B the heart rate has effectively doubled (i.e., increased to 120 bpm), and while the amplitude of potentiation remains large the diastolic time is shorter. In the condition depicted in FIG. 6C, the heart rate is even higher (i.e., at about 150 bpm) and the extrasystole encroaches severely on the cardiac cycle's time in diastole. Furthermore, at these high heart rates PESP potentiation diminishes. The PESP stimulation transforms the 150 bpm tachycardia to a ventricular tachycardia with mechanical alternans and an effective rate of 300 bpm. Heart rates this high are poorly tolerated and will further contribute to cardiac dysfunction, heart failure decompensation, and predispose a person subjected to such an effective heart rate to VT or VF.

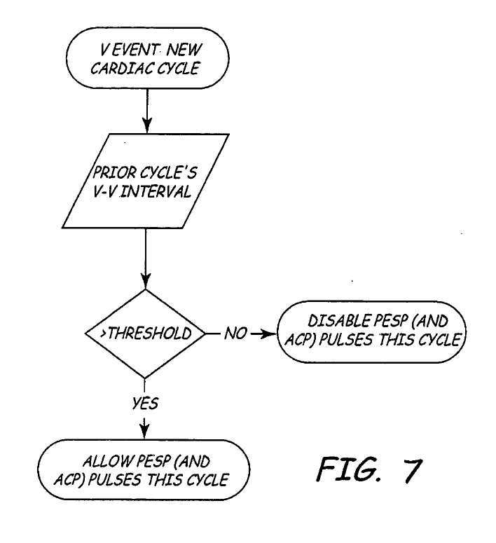

Referring now to FIG. 7, a flow chart for a delivery lockout rule for application of excitatory PESP stimulation is depicted. It can be appreciated that each new cardiac cycle begins with a ventricular event (Vevent) that is either a Vpace or Vsense. The delivery lockout rule has veto power over the decision to deliver excitatory PESP stimulation to the ventricle and possibly atrial coordinated pacing (ACP) during this cycle. If the prior V-V interval is greater than a threshold value, PESP pulses are enabled for this cycle. Should the V-V interval be too short, stimulation therapy is aborted. This prevents stimulation therapy from further adding to the arrhythmic potential of an intrinsic premature ventricular contraction (PVC). Stimulation with a short coupling interval, particularly if immediately following other short intervals can be pro-arrhythmic and is, of course, to be avoided. The delivery lockout rule also prevents application of excitatory therapy during various tachycardias including sinus tachycardia, supraventricular tachycardia (SVT), ventricular tachycardia (VT), or ventricular fibrillation (VF). The threshold used may either be a fixed value or derived from other hemodynamic or electrogram based parameters and is typically 400-600 ms. The delivery lockout rules may operate using a variety of timing schemes which are microprocessor or hardware controlled and programmable with input values determined by algorithms or clinicians, such as depicted in the system diagrams of FIG. 3A and FIG. 3B.

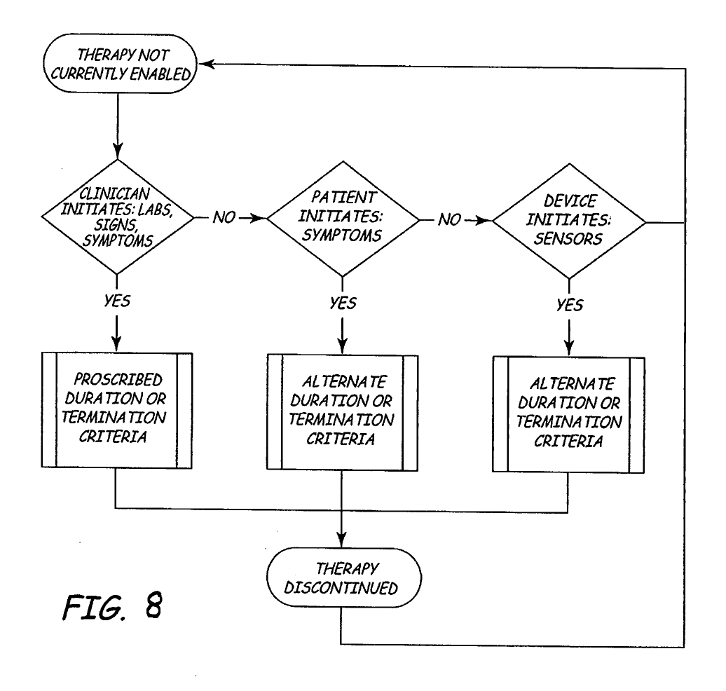

Referring now to FIG. 8, which is a top-level flow chart governing initiation and termination of stimulation therapies according to the embodiments of the invention. If therapy is not currently enabled, a clinician, the patient, or the device can initiate therapy. The clinician is able to preempt an assessment by the device or patient to begin stimulation therapy based on consultation with the patient, signs, or symptoms of cardiac dysfunction, or lab results. If begun in this manner the therapy may have a duration and termination criteria different from patient or device initiated therapy. Similarly, the patient, as a result of symptoms or anticipated exertion may preempt the device and begin therapy. Finally, the device may automatically begin therapy based on preprogrammed time of day or due to sensor signals, including electograms, hemodynamic, activity sensor signals, and other physiologic sensor signals. Therapy may be discontinued by clinician command, patient request, or device based criteria that include sufficient therapy duration and sensor assessment of sufficient benefits or risks.

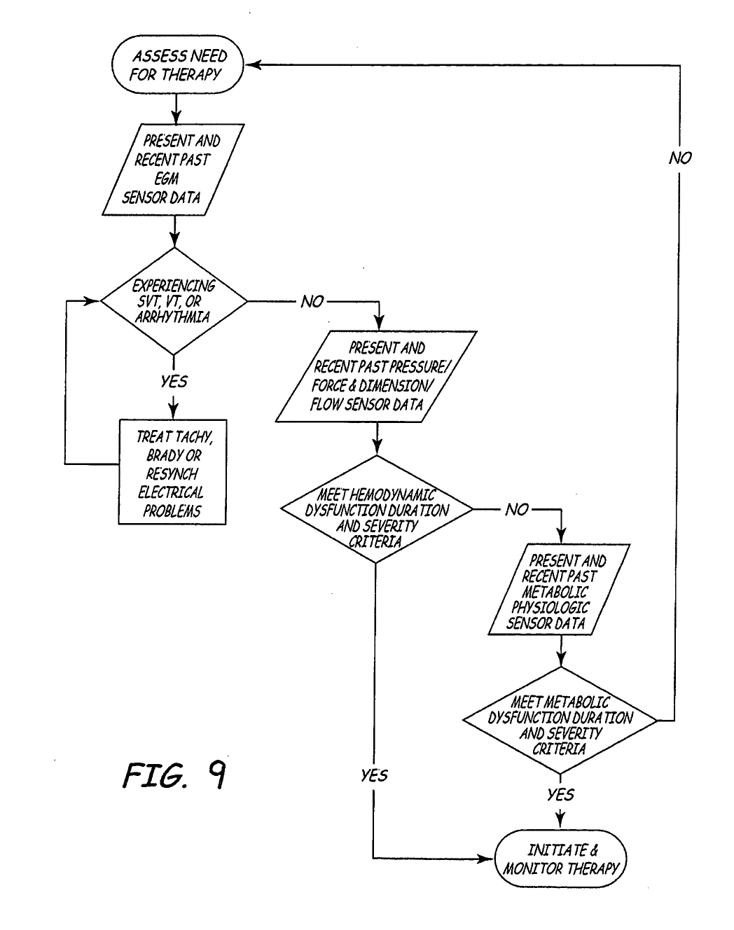

In FIG. 9, a more detailed flow chart of automated sensor-governed initiation of stimulation therapies is shown. PESP therapy could remain on all the time, turned on periodically, or only when sensor indicates therapy is needed. The PESP therapy could also be turned on for so many hours a day. Alternately, the PESP therapy could be based on electrogram (EGM) sensor signals derived from a patient (both presently and recently), the device first looks for and treats cardiac rhythm problems before moving on to examine other sensor signal data. If the cardiac rhythm appears satisfactory, then hemodynamic sensors such as pressure and flow are employed. If there is sufficient dysfunction and duration, therapy begins. Metabolic or other physiologic sensor severity and duration assessments as well as a prescheduled time of day criteria may also initiate stimulation therapies according to the embodiments of the invention.

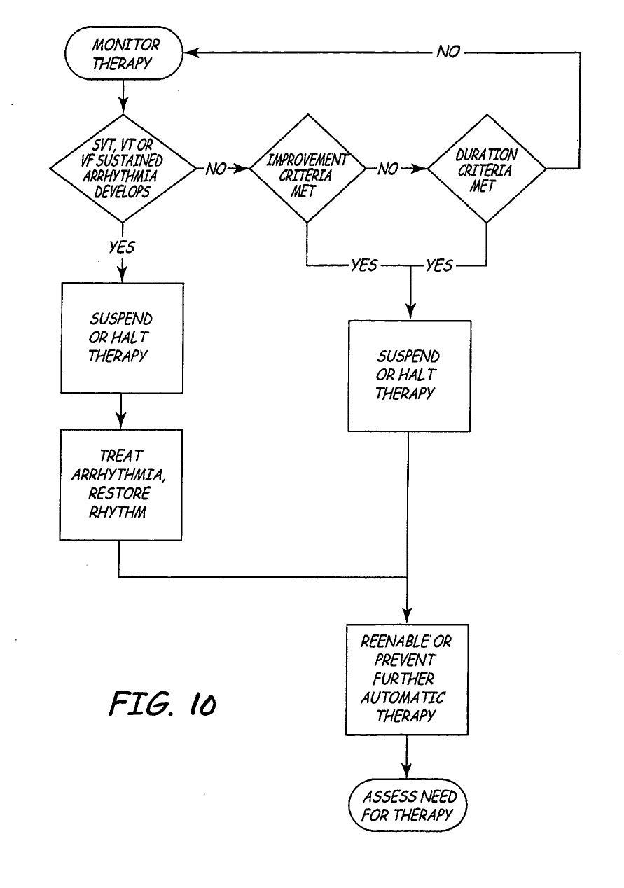

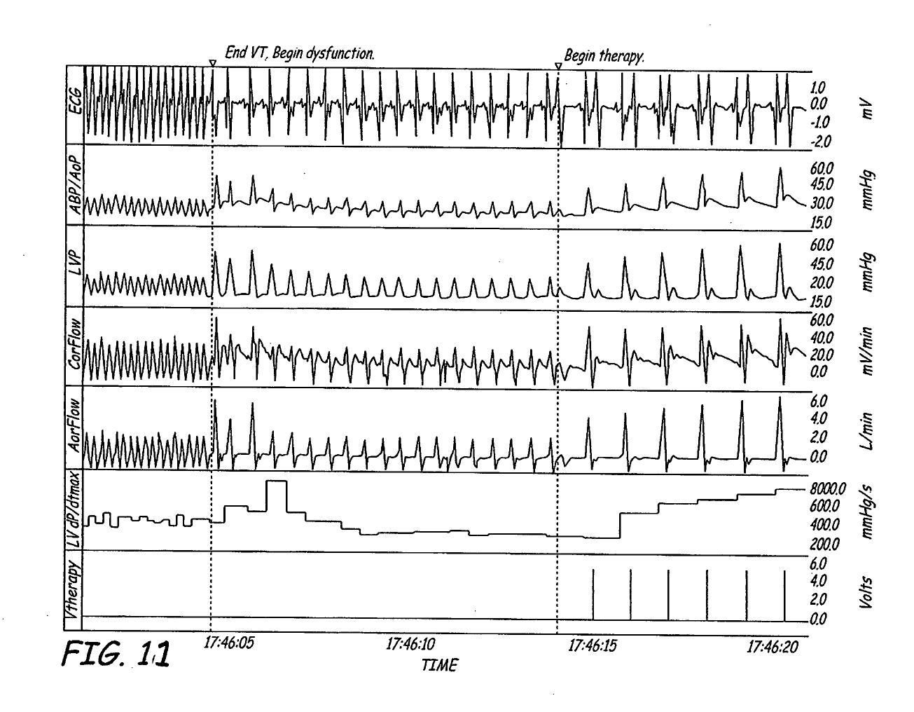

FIG. 10, is an expanded diagram of suspension or termination of stimulation therapies according to an embodiment of the invention. If a tachyarrhythmia develops of sufficient rate or duration (e.g., which exceeds a predetermined rate or duration threshold), the therapy is either temporarily suspended or halted altogether and if necessary, the arrhythmia treated by any of a variety of well-known means such as antitachycardia pacing (ATP), cardioversion, or the like. Upon restoration of a more normal rhythm, the device may or may not re-enable automatic therapy delivery. The device may also readjust its stimulation therapy parameters such as timing and amplitude to achieve a lower arrhythmia risk profile, trading a reduction in physiologic benefit for a reduction in arrhythmia risk (on the presumption that the stimulation therapies either caused or predisposed the subject to this arrhythmia). If the rhythm remains satisfactory, the device checks if either duration or combined hemodynamic improvement and duration criteria are met. If so, the therapies are again either temporarily suspended or halted altogether. Automated therapies may be re-enabled after a period of time or left disabled. In order to prevent multiple brief cyclic applications of therapy, the improvement criteria may be different from the initiation criteria to implement a hysteresis-like effect. Therapies may also be disabled upon reaching a fixed number of therapy applications and require an external override to restart.

Referring now to FIG. 11, which depicts termination of a tachyarrhythmia and initiation of therapy for cardiac dysfunction, FIG. 11 illustrates the therapy initiation rules described above. As can be seen with reference to FIG. 11, a tachyarrhythmia is ended at about 17:46:05 and electrogram sensors (here the surface electrocardiogram (ECG)) confirm the existence of a reasonable rhythm and rate. However, hemodynamic sensors such as arterial blood pressure (ABP) and left ventricular pressure (LVP) confirm a severe level of dysfunction (e.g. LV dP/dtmax<400 mmHg/s) that is sustained for over 6 seconds and over 12 cardiac cycles. As a result, the decision to initiate PESP stimulation therapy occurs at about 17:46:15. A prompt response of arterial blood pressure, LVP, coronary blood flow, aortic blood flow, and LV dP/dtmax is seen coincident with the application of PESP therapy pulses (Vtherapy).

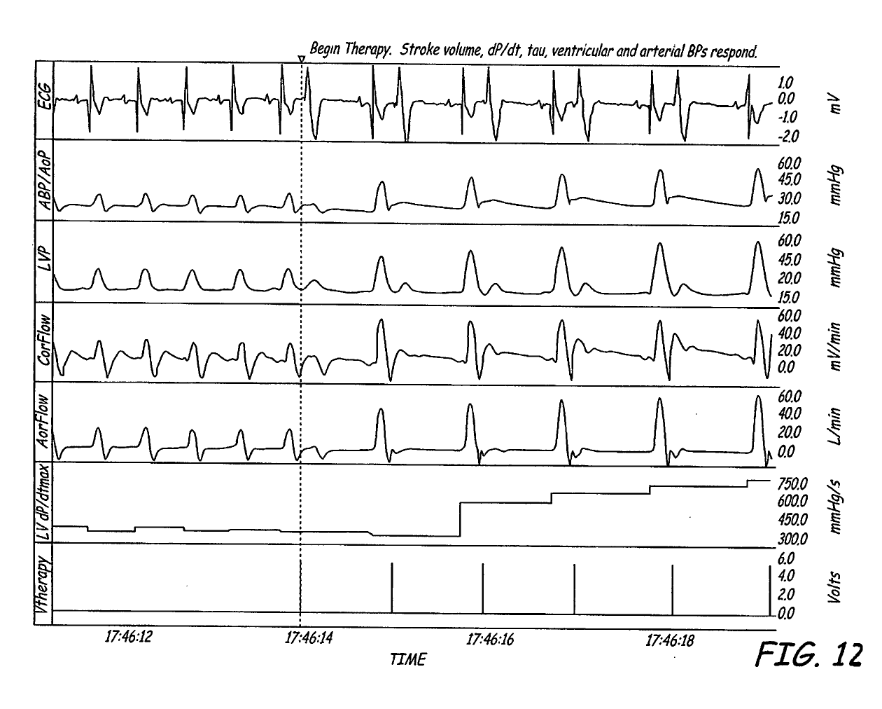

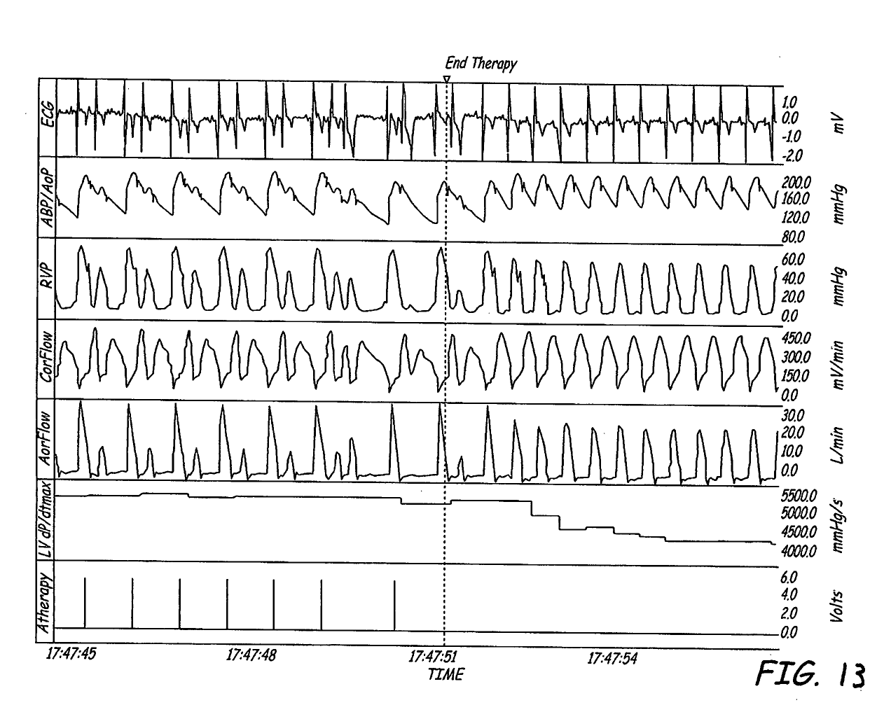

In FIG. 12, an initiation of and response to PESP stimulation therapy is depicted. In other conditions such as HF, not necessarily associated with a preceding or concurrent tachyarrhythmia, cardiac dysfunction may deteriorate to the point where device initiated therapy is required. The onset of such cardiac dysfunction may either be gradual or sudden but upon establishing sufficient severity and duration, PESP stimulation therapy is begun. The excitatory PESP therapy shown here provides much needed increases of arterial blood pressure (ABP), coronary flow (CorFlow) and aortic flow (AorFlow) and the LV dp/dtmax value more than doubles from pre-PESP therapy in approximately five seconds.

FIG. 13 depicts termination of PESP therapy based on duration and response criteria. In FIG. 13, the termination criteria is met and PESP stimulation therapy is halted. In this case, stimulation therapy consists of atrial-only PESP stimulation therapy pulses (Atherapy) which capture and reset the sinus node, are conducted to the ventricles, and produce atrial and ventricular PESP due to natural conduction. In this sequence, the patient has maintained a good RV pressure (RVP) and LV dP/dtmax for over 30-60 seconds, and therefore the atrial-only PESP stimulation therapy is halted. Although the heart rate accelerates and contractility diminishes, cardiac function has recovered very significantly from the levels shown in FIG. 11 and FIG. 12 (just described).

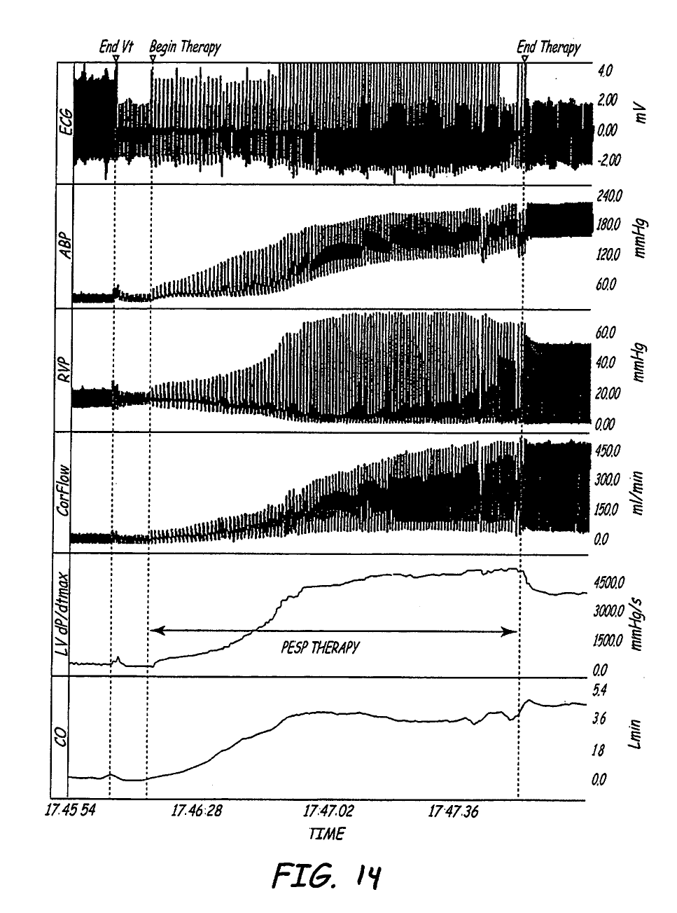

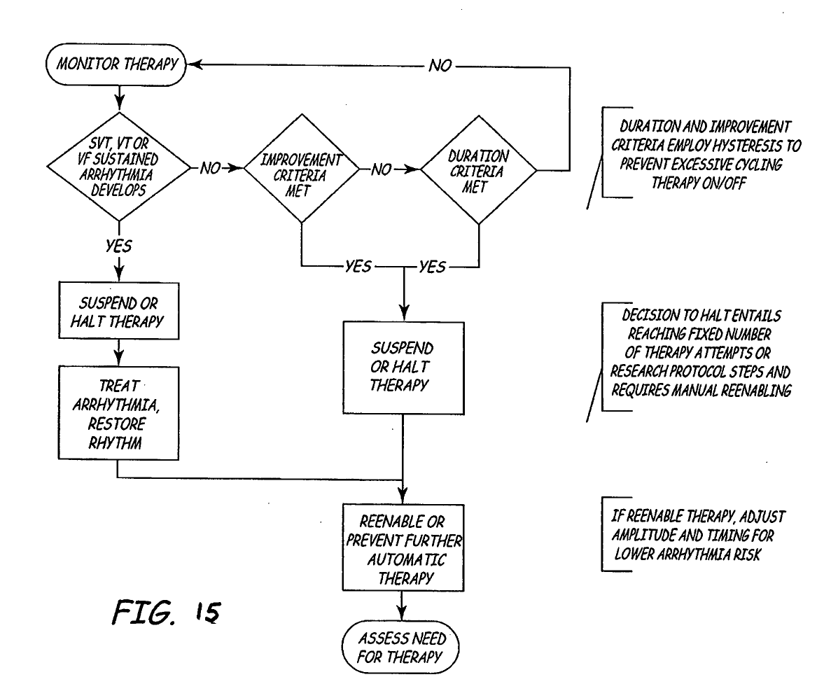

Now turning to FIG. 14 which depicts a dramatic example of lifesaving PESP stimulation therapy. FIG. 14 illustrates (and clearly demonstrates) that post extra-systolic potentiation stimulation therapy can facilitate rapid recovery of cardiac function following a long duration of paced tachyarrhythmia in an anesthetized canine subject.

In FIG. 14, the trace denoted “ECG” is a surface ECG record, the trace denoted “ABP” is a record of arterial blood pressure measured via a catheter in the aorta of the subject, the trace denoted “RVP” is a record of blood pressure measured within the right ventricle. The trace denoted “CorFlow” is a record of blood flow in the coronary artery, the trace denoted “LVdP/dtmax” is a record of the maximum value of the 1^{st} derivative of left ventricular pressure per each cardiac cycle, and the trace denoted “CO” is a recording of cardiac output as derived from mean aortic flow. The record depicted in FIG. 14 begins with the final few seconds of a six-minute long, paced tachyarrhythmia (the portion of the traces before the “End VT” marker). This is followed by approximately 10 seconds of normal sinus rhythm (NSR) with severe hemodynamic dysfunction that could be classified as pulseless electrical activity (PEA) or electro-mechanical dissociation (EMD). During this time, coronary blood flow and cardiac output have not visibly increased compared to flows occurring during the tachyarrhythmia. Without adequate blood flow, the heart will remain ischemic and the subject will likely die of PEA. The portion of FIG. 14 denoted by a horizontal arrow marked “PESP Therapy,” marks the period during which PESP pacing therapies were delivered in the right ventricular apex of the heart of the subject. During this period, all measured pressures and flows are appreciably augmented on the very first cardiac cycle following delivery of the first pacing (PESP) stimuli. The values continue to increase and begin to recover to normal physiologic levels within approximately one minute. At the end of the PESP therapy delivery segment, there has been sufficient coronary flow to re-perfuse the heart, allowing it to resume function without additional therapy. It cannot be overemphasized that return of spontaneous circulation in this subject occurred without any pharmacological or mechanical support therapy or treatment but instead relied exclusively on electrical stimulation delivered according to the embodiments of the invention.

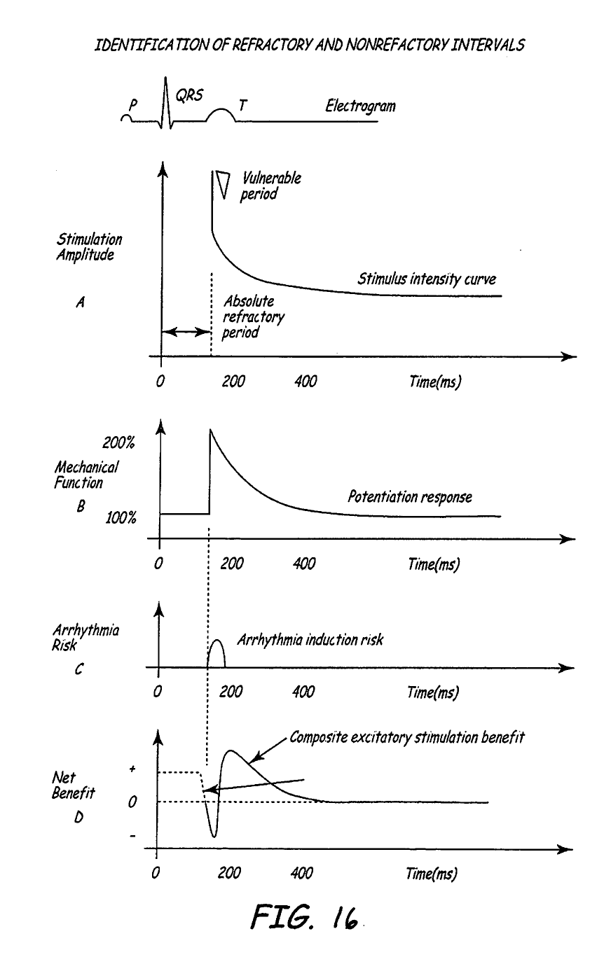

Recognition of the need for such therapy may depend on clinicians or an automated device, either implanted or external, and stimulation therapy applied transcutaneously or from electrodes on or near the heart. FIG. 15, which is an annotated version of FIG. 10, contains some added information regarding duration and improvement criteria, halting therapy delivery and adjustment of amplitude and timing of PESP therapy to lower arrhythmia risk.

The start-stop rules may operate using a variety of schemes and sensor inputs as depicted in FIG. 2 which are microprocessor or hardware controlled and programmable with values determined by algorithms or clinicians, such as depicted in the system diagrams of FIG. 3A and FIG. 3B.

Turning now to FIG. 16 (A through D) which is a composite illustration composed of four X-Y plots of data showing critical timing sequences between such plots of data with respect to delivery of excitatory (PESP) therapy. An unlabeled time-aligned surface representative ECG electrogram trace appears at the top of the figures for ease of cross-reference.

In FIG. 16A, a stimulus intensity curve is depicted wherein a primary determinant of the timing associated with arrhythmia risk and hemodynamic benefit derived from PESP excitatory stimulation. It will be appreciated that stimulation pulses of greater amplitude than the curve (at a given moment in time) are necessary to capture and thus provide benefit from PESP stimulation therapy. An absolute refractory period is depicted in FIG. 16A. During this period no depolarizations result. In the period labeled “vulnerable period,” which occurs just outside of the absolute refractory period, very high amplitude pulses can cause arrhythmias including repetitive extrasystoles, VT, or VF. For practical purposes, excitatory stimulation pulses are delivered some margin above the threshold so that capture is maintained. Stimulation pulse amplitude, however, is also maintained low so that the risk of arrhythmias is very low even when timed to coincide with the vulnerable period (for comparison see FIG. 16C, “arrhythmia induction risk curve”). As is well known in the literature, the magnitude of the potentiation seen on the beat following the extrasystole (the post extrasystole beat) is a function of the extrasystole's timing—becoming greatest at the shortest interval resulting in capture (as shown in FIG. 16B, (labeled “potentiation response” curve). The solid curve depicted in FIG. 16D (labeled “Net Benefit” curve), combines physiologic benefit from excitatory PESP stimulation and arrhythmia risk. It is most desirable to stimulate a little bit longer than (i.e., beyond) the refractory/nonrefractory boundary.

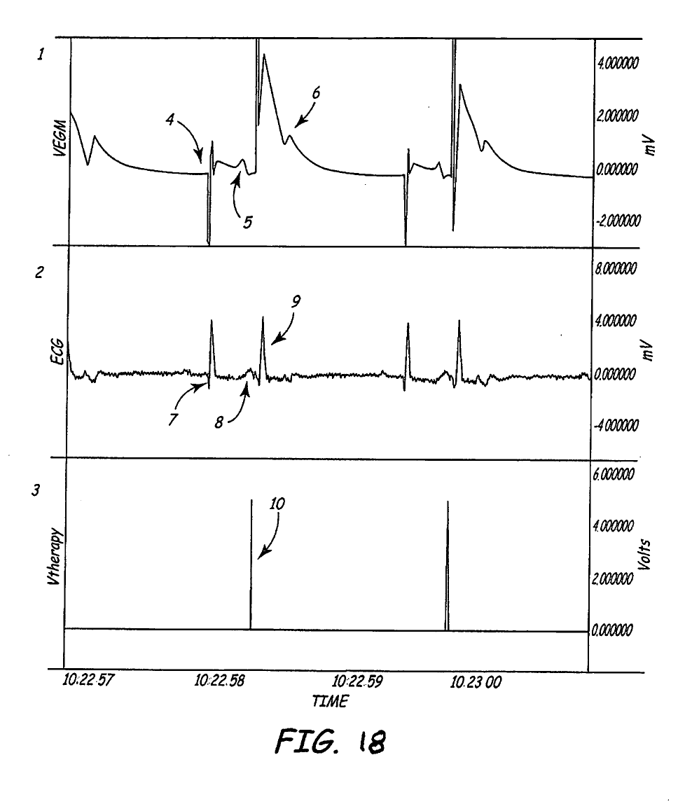

Referring now to FIG. 17, which is a graphical depiction of electrical and hemodynamic detection of cardiac chamber capture. The trace labeled “1” is a ventricular electrogram (VEGM) obtained from the site of application of the stimulation therapy. The trace labeled “2” is a second electrogram that is near both right atrium and right ventricle and is away from the site of application of the pacing therapy. The trace labeled “3” is a surface ECG, trace “4” is a record of arterial blood pressure (ABP), trace “5” is a record of left ventricular pressure (LVP), trace “6” is a record of right ventricular pressure (RVP) and trace “7” is a marker channel record of stimulation therapies applied to the ventricles (Vtherapy). FIG. 17 illustrates embodiments of the concept of the identification of whether or not a cardiac potentiation therapy lies inside or outside the cardiac refractory period.

With respect trace 7, arrow 219 identifies a therapy is delivered to the ventricle that lies inside the refractory period, arrow 220 identifies a therapy that lies outside the refractory period. With respect to trace 1, arrow 208 identifies an electrogram tracing following a therapy that shows no evidence of a resultant depolarization, confirming that the therapy lies in the refractory period, and arrow 209 identifies an electrogram tracing showing a cardiac depolarization following the therapy, confirming that the therapy pulse, had sufficient amplitude and duration, was outside the refractory period, and captured the myocardium.

Similarly, with respect to trace 2, arrows 210 and 211 identify noncapture and capture, respectively, from the electrogram at an auxiliary electrode site suitable to identify pulses inside and outside of the cardiac refractory period by the absence or presence of a ventricular depolarization. With respect to trace 3, arrows 212 and 213 identify the absence and presence of ventricular depolarizations on a surface ECG, respectively.

An embodiment of the invention would be to apply a detection algorithm to electrogram signals recorded at one or more sites where PESP is delivered (possibly including but not limited to signal traces 1-3) and identifying the presence or absence of an evoked depolarization. This information is then used to identify whether the preceding therapy was inside or outside of the cardiac refractory period.

With respect to trace 4, arrow 214 points to a significantly augmented ABP wherein the arterial pulse pressure was augmented on the cardiac cycle following a therapy that lies outside the refractory period. Similarly, LVP (trace 5) and RVP (trace 6) are also augmented on the cycle following capture. Thus, FIG. 17 illustrates an embodiment of these embodiments of the invention used to detect the presence of pressure, flow, acceleration, impedance change, or other favorable evidence of mechanical augmentation following therapy delivery. This evidence also helps identify whether or not the preceding therapy was delivered inside or outside of the cardiac refractory period.

With respect to traces 5 and 6, arrows 215 and 217 indicate portions of a left and right ventricular pressure waveform, respectively, resulting from stimulation therapy delivered in the cardiac refractory period. As a result, no evidence of an extrasystole is seen following the therapy.

Again with respect to traces 5 and 6, arrows 216 and 218 are pressure waveforms following a therapy delivered outside of the cardiac refractory period. An extrasystole can be seen following this therapy. Another embodiment of the invention is adapted to apply a detection algorithm to a sensor that makes a measurement of cardiac mechanical activity, including but not limited to right ventricular, left ventricular or arterial pressure, dimension, or acceleration and identifying the presence or absence of an extrasystole. This information is used to identify whether the preceding therapy was inside or outside of the cardiac refractory period. Evoked R wave detection information may then be used to time or trigger delivery of a stimulation therapy that would cause post extra-systolic potentiation.

FIG. 18 depicts three traces, VEGM, ECG and Vtherapy, respectively which can be used to determine whether or not capture has occurred by analyzing a T wave. Trace 1 is a ventricular electrogram (VEGM) from the site of application of the stimulation therapy, trace 2 is a surface ECG, and trace 3 is a marker channel record of applied stimulation therapies. With respect to trace 1 and 2, arrows 4 and 7 are electrogram signals indicating a ventricular depolarization and arrows 5 and 8 are signals showing a resulting ventricular repolarization or T-wave. In trace 3, arrow 10 corresponds to a marker of the delivered therapy, which was applied just after the T-wave. In traces 1 and 2, arrows 6 and 9 are an evoked T resulting from repolarization.

FIG. 18 depicts three traces, VEGM, ECG and Vtherapy, respectively which can be used to determine whether or not capture has occurred by analyzing a T wave. Trace 1 is a ventricular electrogram (VEGM) from the site of application of the stimulation therapy, trace 2 is a surface ECG, and trace 3 is a marker channel record of applied stimulation therapies. With respect to trace 1 and 2, arrows 4 and 7 are electrogram signals indicating a ventricular depolarization and arrows 5 and 8 are signals showing a resulting ventricular repolarization or T-wave. In trace 3, arrow 10 corresponds to a marker of the delivered therapy, which was applied just after the T-wave. In traces 1 and 2, arrows 6 and 9 are depolarizations evoked from the therapy pulse.

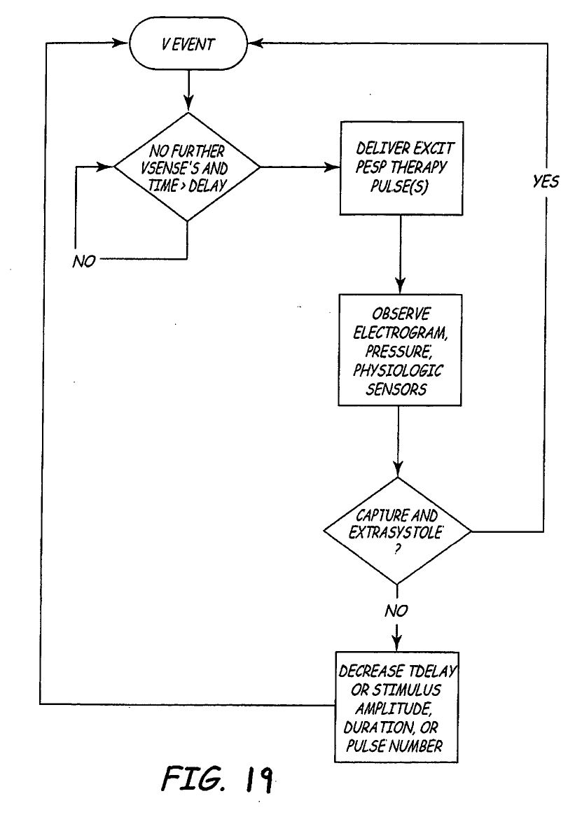

FIG. 19 is a flow chart that diagrams response to capture information to apply excitatory PESP therapy. Following a ventricular pace or sense event, the sensing circuits such as depicted in FIG. 3A and FIG. 3B remain active and a timer counts down a delay until the scheduled delivery of the PESP stimulation pulse(s). If there has been no intrinsic event in this interval, the pulse(s) are delivered and electrogram or mechanical sensor signals employed (such as described herein above) to determine if capture and an extrasystole occurred. If capture did not occur, the delivery time, stimulation amplitude, or pulse number is increased and the process repeated. The value for ESI (here referred to as Tdelay) is typically 200-300 ms. Tdelay and other stimulus parameters may also be influenced by observations of heart rate or other physiologic sensors in addition to the electrical and mechanical parameters discussed above.

The identification of refractory and non-refractory intervals and appropriate timing of pulses may operate using a variety of timing schemes and sensing circuits which are both preferably microprocessor or hardware controlled and programmable with input values determined by algorithms or clinicians, such as depicted in the system diagrams of FIG. 3A and FIG. 3B.

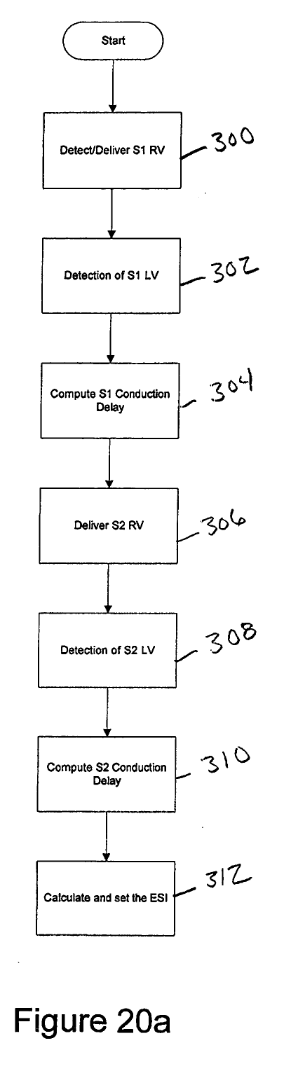

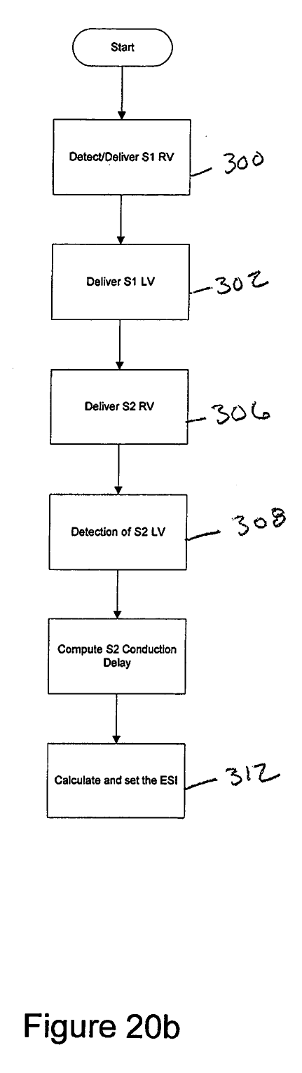

With reference to FIG. 20a, a flow diagram is shown of a program for determining and controlling the ESI in an embodiment of the embodiments of the invention. The embodiments of the invention enhance PESP therapy by expanding it to multiple ventricular sites and using information acquired from multiple ventricular sites and conduction times between the multiple sites to control its application at the multiple sites. It provides a method for controlling the ESIs, which is believed to be helpful for optimal and safe delivery of PESP therapy. The embodiments of the invention also provide methods for delivery of PESP therapy at multiple sites to ensure uniformity and maximal effect of PESP therapy delivery throughout the heart. As discussed above, it is helpful to provide the PESP therapy at each individual site as close to the site specific refractory period as possible to obtain a greater contraction on the next pulse. Therefore, it is helpful to set the ESI so that the PESP therapy is delivered at the optimal time at each site in the heart. FIG. 20 details a method for determining an optimal ESI at one or both sites through the monitoring of the conduction times between sites for both the sensed and paced events. Typically this would occur in the ventricular chambers, RV and LV. However, detection and control of the ESI could occur between the atrium and the ventricular chamber, at multiple LV sites, or with three or more sites.

FIG. 20a depicts an evaluation of conduction times between multiple ventricular sites to calculate a site specific ESI. This algorithm could be run periodically (e.g. daily, hourly, every 30 beats) or upon detection of a change in physiologic state based on sensor feedback (e.g., increase/decreased hemodynamic performance, increased/decreased heart rate, increased/decreased activity, etc), thus allowing the automatic adjustment of site specific ESIs based on the physiologic milieu. The locations of the two sites are preferably the RV and LV, but could be extended to any two locations within the heart (ventricular or atrial) and could easily be extended to more than two sites by someone skilled in the art. Initially an S1 event (paced or intrinsic) occurs at an RV site (state 300) and is detected at an LV site shown at state 302. IMD 14 then computes the S1 conduction delay at state 304. The S1 event will conduct from the RV site to the LV site in a certain amount of time, the S1 conduction delay (e.g., 20 milliseconds). The S2 coupled or paired pace delivered at the RV site (state 306) is also detected at the LV site after a certain S2 conduction delay (e.g., 30 milliseconds) as shown in state 308. IMD 14 then computes the S2 conduction delay at state 310. It is believed that the S2 conduction delay will be greater than the S1 conduction delay because of the prematurity of the S2. In addition, the S2 conduction delay is believed to be related to the pre-maturity of the S2 pace, with greater prematurity leading to larger S2 conduction delays. Based upon the conduction times from the RV site to the LV site for both the S1 and S2 beats, the ESI can be set at state 312.

Note that if the S1 RV event is a sense, a further adjustment may need to be made to the computation of the LV ESI because the conduction delays will be different for the sensed S1 beat and the paced S2 beat. One example of such an adjustment would be to add a constant value to the sensed S1 conduction time. This value may be programmed by the clinician and/or determined automatically or manually by subtracting a paced RV S1 conduction time to the LV from a sensed RV S1 conduction time to the LV. This constant offset value could also be modified by the rate. An algorithm can then be used to calculate and set the ESI times for each site, depending on the desired outcome. In one embodiment, the LV ESI is computed as a function of the RV ESI and the S1 or S2 conduction delay as illustrated in FIG. 20b. One implementation of such an embodiment computes the LV ESI as the RV ESI plus a value equal to a constant times the S2 conduction delay. Thus, as the conduction delay increases between the RV and LV, the ESI at the LV will increase proportionally. Note that the S1 conduction delay could be used in lieu of the S2 conduction delay. In another embodiment, the LV ESI is a function of the RV ESI, the S1 conduction delay, and the S2 conduction delay. In one implementation of such an embodiment, the LV ESI equals the RV ESI times the ratio of the S2 conduction delay and the S1 conduction delay. Therefore, the LV ESI would increase as the S2 conduction delay increases with respect to the S1 conduction delay.

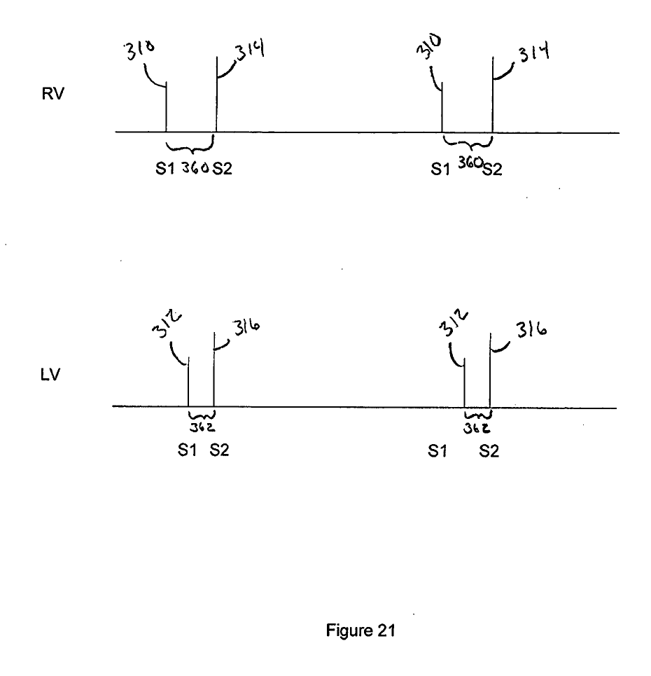

With reference to FIGS. 21-28, marker channel diagrams of various PESP delivery methods in embodiments of the present invention are shown. Shorter lines indicate sensed intrinsic depolarizations while relatively longer lines indicate delivered paces. With the ability to determine and set the ESI, as discussed above, several embodiments of delivery of PESP at one or more ventricular sites can be achieved to ensure uniformity of PESP therapy delivery around the heart. With reference to FIG. 21, a simultaneous extra-systole to the right and left ventricle is described. As shown, RV S1 sense 310 occurs followed by LV S1 sense 312. A sensed event is defined as the sensing of a natural depolarization or a pacing event of the ventricle or atrium. RV PESP pulse S2 314 can then be delivered after RV ESI 360, which can be predetermined by the implanting clinician, set after implantation through uplink programming as discussed above, or can be set by monitor/sensor 100 after detection of the refractory period. In this case, the LV ESI 362 is determined and set so the LVS2 pulse 316 is simultaneous with the RVS2 pulse 314, with LV ESI=RV ESI+(LVS1−RVS1). In this embodiment RV ESI 360 is not the same as the LV ESI 362. That is, RV S2 pace 314 may be coupled to RV S1 sense 310 a certain ESI following the RV S1 sense 310. The LV S2 pace 316 is delivered simultaneously in this embodiment with the RV S2 pace 314. Of course, it is understood that in other embodiments the S2 pace may be triggered off of the LV S1 sense. In order to ensure that S2 capture occurs at both sites, it may be necessary to monitor each site for an evoked response. If loss of capture occurs at the LV site and not the RV site, the ESI at the LV site will need to be extended, thereby extending the ESI at the LV site. If this does not work, a different multi-site PESP embodiment may be needed (described below), or the device could switch to a single site PESP mode. The advantage of this embodiment is its easy implementation, delivering PESP at multiple sites and requiring only a single ESI for both sites.

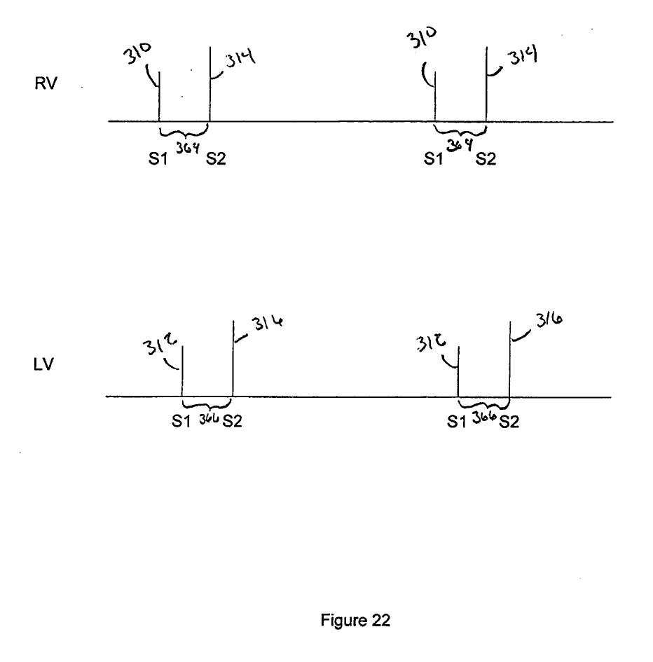

With reference to FIG. 22, a marker channel diagram of a sequential extra-systole to the right and left ventricle in an embodiment of the present invention is shown. As above, RV S1 sense 310 occurs and is followed by LV S1 sense 312. Similar to above, RV PESP pulse S2 314 can then be delivered after the RV ESI 364, which can be predetermined by the implanting clinician, set after implantation through uplink programming as discussed above, or can be set by monitor/sensor 100 after detection of the refractory period. In this embodiment, however, RV ESI 364 is generally equal to LV ESI 366 so that pulse 314 and LV PESP pulse S2 316 occur sequentially. That is, LV S2 pace 316 is delivered after RV S2 pace 314 based upon LV ESI 366 being relatively equal to RV ESI 364. Similar to the embodiment of FIG. 21, the RV and LV sites can be monitored for S2 capture, resulting in extension of the RV and LV ESIs if capture is lost at one or both sites. This embodiment is easily implemented, requiring only a single ESI for both sites, yet resulting in a more synchronous spread of the S2 wavefront throughout the heart.

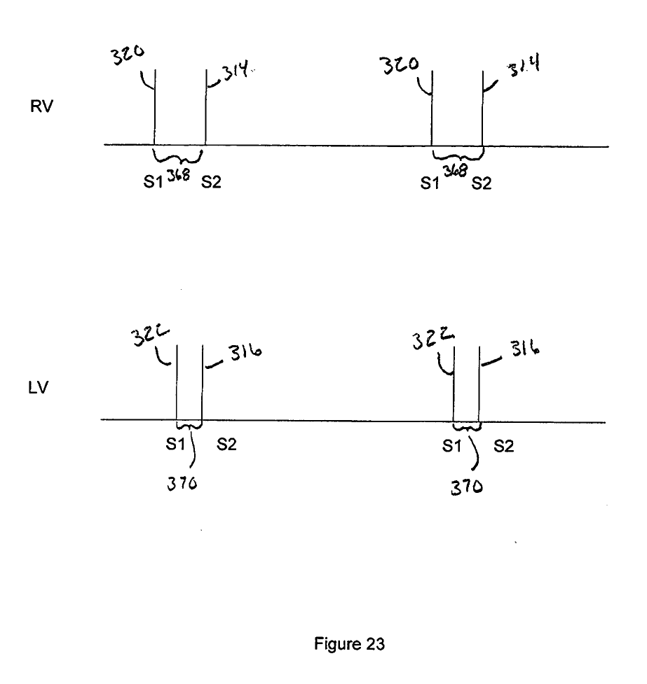

With reference to FIG. 23, a marker channel diagram of a sequential pacing of the right and left ventricle with simultaneous extra-systoles to the right and left ventricle in an embodiment of the present invention. As shown, RV S1 pace 320 is delivered first and is followed by LV S1 pace 322 delivered immediately or at a predetermined time after pace 320 (e.g., typically 0 ms to 40 ms). Similar to above, RV PESP pulse S2 314 can then be delivered after an RV ESI 368, which can be predetermined by the implanting clinician, set after implantation through uplink programming as discussed above, or can be set by monitor/sensor 100 after detection of the refractory period. In this Figure, RV ESI 368 is not equal to LV ESI 370 so that pulse 314 and LV PESP pulse S2 316 occur simultaneously, but the RV and LV S2 pulses could be delivered sequentially as well. This embodiment provides a form of cardiac resynchronization for the S1 beat as well as a more uniformly propagated wavefront for the S2 beat, leading to a more uniform delivery of PESP.

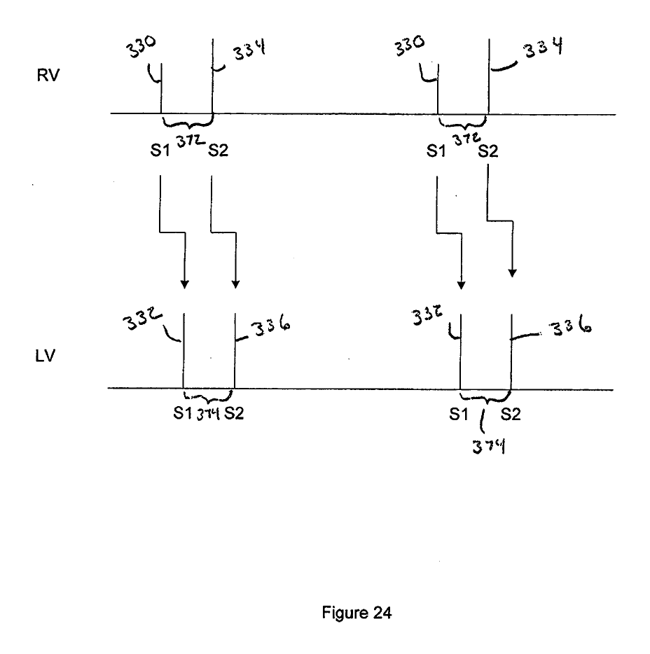

With reference to FIG. 24, a marker channel diagram of an extra-systole delivered to the right and left ventricle based upon a triggering event in the RV in an embodiment of the present invention is shown. As shown, RV S1 sense 330 triggers LV S1 pace 332. RV PESP pulse S2 334 then also triggers LV PESP pulse S2 336. Similar to above, RV PESP pulse S2 334 can then be delivered after the RV ESI 372, which can be predetermined by the implanting clinician, set after implantation through uplink programming as discussed above, or can be set by monitor/sensor 100 after detection of the refractory period. In this embodiment, LV ESI 374 is derived from the RV ESI 372, taking into account an assumed or previously measured conduction delay between the RV and LV. The delay between 330 and 332 and between 334 and 336 may be the same, or different, based on the desired clinician programming. This embodiment may be useful in that the triggered S1 in the LV synchronizes the electrical and mechanical wavefront movement throughout the ventricle. Furthermore, the computation of the LV ESI based upon the RV ESI and information about the variable conduction delays between the RV and LV ensures a more uniform spread of the electrical wavefronts associated with the S2.

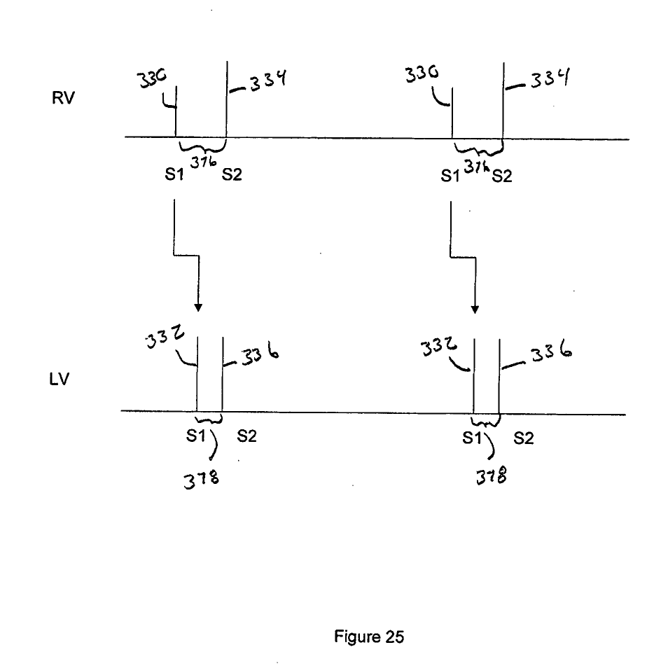

In another embodiment shown in FIG. 25, RV S1 sense 330 triggers LV S1 pace 332. S2 paces 334 and 336 could occur simultaneously based upon an ESI following the RV S1 sense 330. RV PESP pulse S2 334 can then be delivered after RV ESI 376, which can be predetermined by the implanting clinician, set after implantation through uplink programming as discussed above, or can be set by monitor/sensor 100 after detection of the refractory period. An LV ESI 378 can also be determined and set utilizing the method discussed above so that pulse 334 and LV PESP pulse S2 336 occur simultaneously. In this embodiment RV ESI 376 is not the same as the LV ESI 378. That is, RV S2 pace 334 may be coupled to RV S1 sense 330 at a certain ESI following the RV S1 sense 330. The LV S2 pace 336 is delivered simultaneously in this embodiment with the RV S2 pace 334. This embodiment may be useful in that the triggered S1 in the LV synchronizes the electrical and mechanical wavefront movement throughout the ventricle.

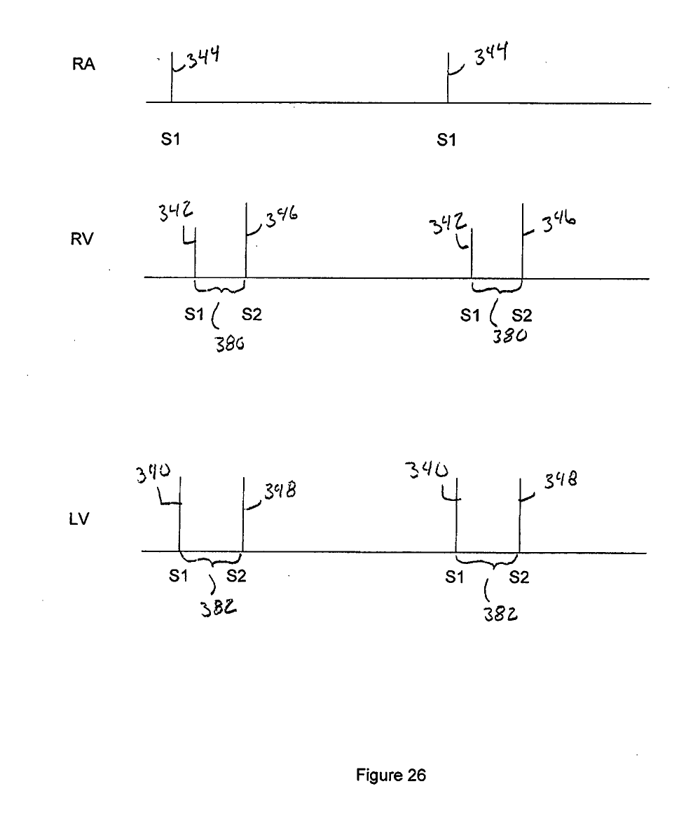

With reference to FIG. 26, a marker channel diagram of a simultaneous extra-systole delivered to the right and left ventricle with fusion pacing of the ventricles in an embodiment of the present invention is shown. In certain patients exhibiting symptoms resulting from congestive heart failure (CHF), cardiac output is enhanced by timing the delivery of LV pacing pulse S1 340 such that the evoked electrical depolarization and mechanical contraction of the LV results in a fusion with the intrinsic RV S1 342 evoked depolarization and contraction. The resultant fusion enhances stroke volume in such hearts where the intrinsic atrio-ventricular (AV) conduction of a preceding atrial depolarization wave front S1 344 (intrinsic or evoked) conducts to the RV and is sensed (S1 342), but wherein the AV conducted depolarization to the LV is unduly delayed. The fusion depolarization of the LV is attained by timing the delivery of the LV pace pulse S1 340 to occur before the intrinsic depolarization of the RV and precede the intrinsic depolarization of the LV. Specifically, an RV pace pulse is not delivered, allowing natural propagation of the wave front and depolarization of the RV and intraventricular septum, while LV pulse S1 340 is delivered to fusion with RV depolarization S1 342. RV PESP pulse S2 346 can then be delivered after RV ESI 380. LV ESI 382 can also be determined and set utilizing the methods discussed above to obtain simultaneous PESP S2 pacing. In this embodiment RV ESI 380 is generally unequal to LV ESI 382, resulting in simultaneous PESP S2 paces unless RV S1 342 and LV S1 340 occur simultaneously. This embodiment allows for a more natural contraction of the RV, allows for intrinsic AV interval modulation, and requires less energy for the S1 beat, while delivering a simultaneous S2 beat to both the RV and LV.

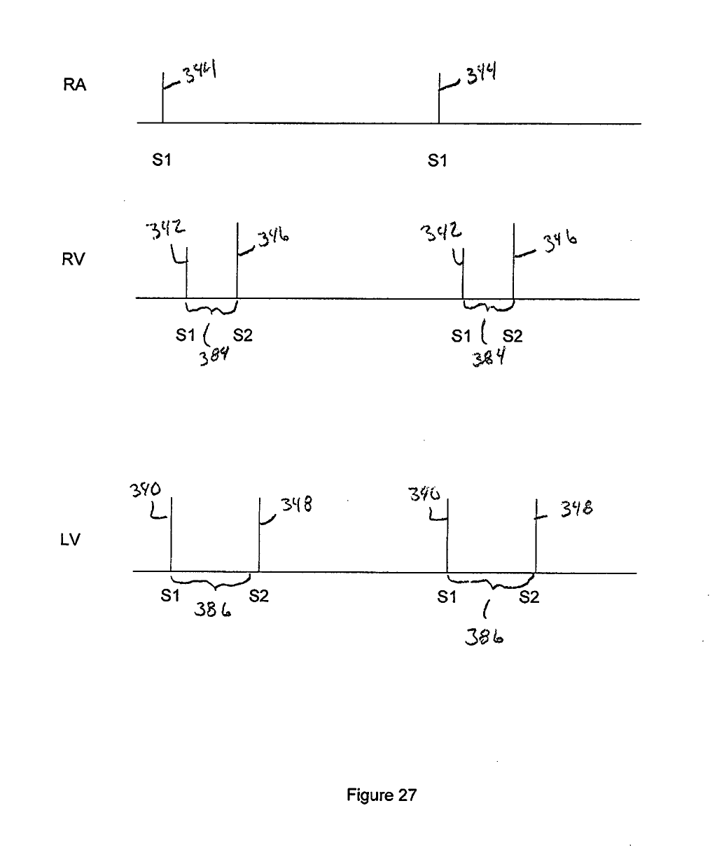

Similar to the embodiment shown in FIG. 26, FIG. 27 shows a waveform diagram of a sequential extra-systole delivered to the right and left ventricle with fusion pacing of the ventricles in an embodiment of the present invention. In this illustration, PESP paces 346 and 348 are sequential. Similar to above, RV PESP pulse S2 346 can be delivered after the RV ESI 384, which can be predetermined by the implanting clinician, set after implantation through uplink programming as discussed above, or can be set by monitor/sensor 100 after detection of the refractory period. In this embodiment, LV ESI 386 is generally equal to RV ESI 384 so that pulse 346 and LV PESP pulse S2 348 occur sequentially. That is, LV S2 pace 348 is delivered after RV S2 pace 346 based upon LV ESI 386 being relatively equal to RV ESI 384. Thus a more synchronized cardiac augmentation can be realized through Fusion pacing only a single ventricular site for the S1 and delivering an S2 specifically timed for each ventricular site based upon the local tissue properties as compared to the traditionally described PESP.

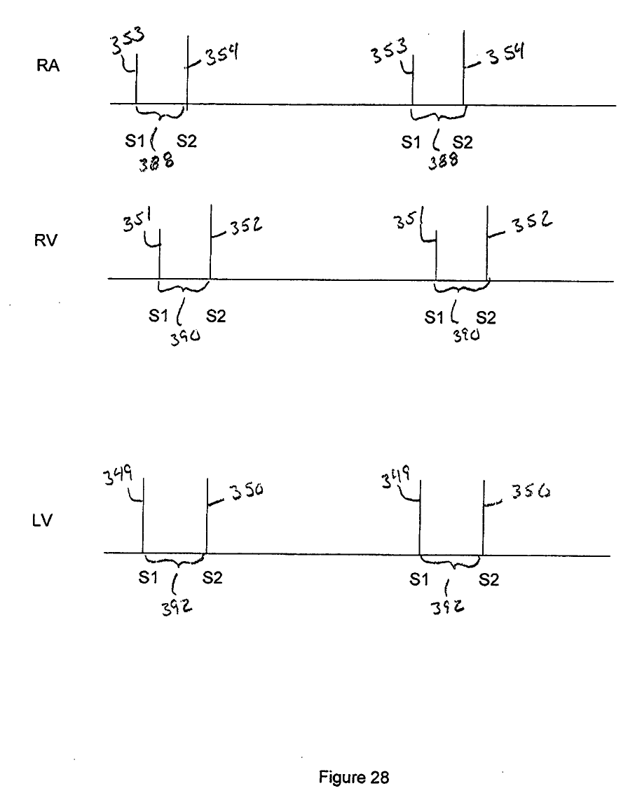

With reference to FIG. 28, a waveform diagram of an extra-systole delivered to the right atrium, right ventricle, and left ventricle with PESP fusion pacing of the ventricles in an embodiment of the present invention is shown. The delivery of the RV PESP and LV PESP can be based off of an RA PESP pulse such that depolarization of the LV is effected prior to, at the same time, or just after the RV PESP similar to the fusion pacing embodiments of FIGS. 26 and 27 above. The delivery of LV pacing pulse S1 349 can be linked to the intrinsic RV depolarization S1 351, with LV S1 349 occurring before or after RV S1 351. The RV depolarizes first due to intact atrio-ventricular (AV) conduction of a preceding intrinsic or evoked atrial depolarization wave RA S1 353, but wherein the AV conducted depolarization of the LV is unduly delayed. The depolarization of the LV is attained by timing the delivery of the LV pace pulse S1 349 to occur before or follow the intrinsic depolarization of the RV but to precede the intrinsic depolarization of the LV.

RA S2 pulse 354 is delivered after the atrial ESI 388 which can be predetermined by the implanting clinician, set after implantation through uplink programming as discussed above, or can be set by monitor/sensor 100 after detection of the refractory period. RV S2 pulse 352 could then be administered after ESI 390 corresponding to a time corresponding to the AV conduction time. The delivery of LV PESP pulse S2 350 could then be dependant on ESI 392 corresponding to a time prior to, at the same time, or just after the RV PESP S2 pulse 352 dependant on which method would effect optimum fusion of the PESP pulses thereby ensuring optimum PESP pacing and depolarization. This method provides an enhanced atrial contribution to the enhanced ventricular contraction, thus potentially providing an even greater PESP effect.

It is noted that the timing of all the S1 and S2 pulses in each of the locations can all be determined based on sensed or paced events at any location in the heart. For example, in fusion pacing (FIGS. 26-28), the setting of the S1 pace in the LV, the LV is paced before the RV. Therefore, methods are used that track the intrinsic A-RV time and then set an A-LV interval for LV pacing. Therefore, the LV pacing is timed off the RA sense and the previous history of A-RV senses. If fusion pacing is not occurring, then the timing of the S1 at the RV (and LV) sites could be based off the atrial event, RV event, or any other cardiac event. In the case of the S2, it can be delivered at the RV site based on an ESI or based on a timer expiring from the S1 event in the LV. There are many permutations here, with reference to FIG. 26, as an example, where T_{SX} refers to the time of an event X at site S. In the case of Fusion pacing, the paced LV_{S1} occurs before the sensed RV_{S1} and is determined based upon the history of previous RA-RV conduction times. Therefore, the ith beat uses information from the (i-1)th beat (and possibly even more previous beats), leading to the following formulation:

T_{LVS1}^{(i)=T}_{RAS1}⁽ⁱ⁾+((T_{RAS1}⁽ⁱ⁻¹⁾−T_{RAS1}⁽ⁱ⁻¹⁾−Δ), where Δ is the pre-excitation

interval describing how much before the RV sense the LV should be paced.

A more general notation can be used throughout FIGS. 21-28: T_{RVS2}=(T_{RVS1}+ESI), where ESI is a function of output from a hemodynamic sensor, rate, and/or mechanical sensor. Yet another example notation could be T_{LVS2}=f(T_{RVS1}, T_{LVS1}, T_{RVS2}, sensor input). There are obviously many permutations of these timing relationships.

Thus, embodiments of MULTI-SITE PESP WITH FUSION PACING are disclosed. One skilled in the art will appreciate that the present invention can be practiced with embodiments other than those disclosed. The disclosed embodiments are presented for purposes of illustration and not limitation, and the present invention is limited only by the claims.

What is claimed is:

1. A method of providing coupled ventricular pacing, comprising:
sensing ventricular events at a first ventricular site and generating a ventricular sense event signal in response thereto; and
providing coupled pacing pulses simultaneously at the first ventricular site and at a second ventricular site at a ventricular extra stimulus interval (VESI) timed from immediately preceding ventricular sense event signals sufficient to effect post-extra-systolic potentiation (PESP) of the ventricular sites.

2. The method of claim 1, wherein the ventricular events sensed at the first ventricular site are intrinsic ventricular depolarizations.

3. The method of claim 1, wherein the ventricular events sensed at the first ventricular site are paced events.

4. The method of claim 1, wherein the first ventricular site is located in one ventricle and the second ventricular site is located in the other ventricle.

5. The method of claim 1, further including providing pacing pulse at the second ventricular site after sensing ventricular events at the first ventricular site.

6. The method of claim 5, wherein the pacing pulse at the second ventricular site is provided sequentially following sensed ventricular events at the first ventricular site.

7. The method of claim 5, wherein the pacing pulse is timed such that depolarization waves resulting from ventricular depolarizations at the first and second ventricular sites fuse together at some intermediate location between the first and second ventricular sites.

8. The method of claim 1, further including sensing atrial events at an atrial site, providing a ventricular pacing pulse at the second ventricular site following the sensed atrial event such that depolarization waves resulting from intrinsic ventricular depolarizations at the first ventricular site and paced depolarizations at the second ventricular site fuse together at some intermediate location between the first and second ventricular sites.

9. The method of claim 1, wherein the VESI is automatically adjusted to ensure PESP at both sites.

10. The method of claim 1, wherein the PESP is turned off at one or both ventricular sites if capture cannot be obtained at one or both ventricular sites with the same ESI.

11. A method of operating an implantable cardiac pacing device to provide coupled ventricular pacing, comprising:
sensing ventricular events at a first ventricular site and generating a first ventricular sense event signal in response thereto;
sensing ventricular events at a second ventricular site and generating a second ventricular sense event signal in response thereto;
providing pacing pulses at the first ventricular site at a ventricular extra stimulus interval (VESI) timed from immediately preceding first ventricular sense event signals sufficient to effect post-extra-systolic potentiation (PESP) of the first ventricular site; and
providing a pacing pulse at the second ventricular site at the VESI sufficient to effect PESP of the second ventricular site.

12. The method of claim 11, wherein the ventricular events sensed at the first ventricular site are intrinsic ventricular depolarizations.

13. The method of claim 11, wherein the first ventricular site is in one ventricle and the second ventricular site is in the other ventricle.

14. The method of claim 11, further including providing pacing pulses at the second ventricular site after sensing ventricular events at the first ventricular site.

15. The method of claim 14, wherein the pacing pulses at the second ventricular site are provided sequentially following sensed ventricular events at the first ventricular site.

16. The method of claim 14, wherein the pacing pulses are timed such that depolarization waves resulting from ventricular depolarizations at the first and second ventricular sites fuse together at some intermediate location between the first and second ventricular sites.

17. The method of claim 11, further including sensing atrial events at an atrial site, providing a ventricular pacing pulse at the second ventricular site following the sensed atrial event such that depolarization waves resulting from intrinsic ventricular depolarizations at the first ventricular site and paced depolarizations at the second ventricular site fuse together at some intermediate location between the first and second ventricular sites.

18. A method of operating an implantable cardiac pacing device to provide coupled pacing, comprising:
sensing intrinsic atrial depolarizations at an atrial site and generating an atrial sense event signal in response thereto;
sensing intrinsic ventricular depolarizations at a first ventricular site and generating a first ventricular sense event signal in response thereto;
providing coupled pacing pulses at the atrial site at an atrial extra stimulus interval (AESI) timed from the atrial sense event signal sufficient to effect post-extra-systolic potentiation (PESP) of the atrium;
sensing intrinsic ventricular depolarizations at the first ventricular site following immediately preceding coupled pacing pulses at the atrial site and generating a second ventricular sense event signal in response thereto; and
providing coupled pacing pulses at a second ventricular site at a ventricular extra stimulus interval (VESI) timed from one of the immediately preceding second ventricular sense event signal and the immediately preceding atrial sense event signal sufficient to effect PESP of the second ventricular site.

19. The method of claim 18, wherein the VESI is set such that depolarization waves resulting from ventricular depolarizations at the first and second ventricular sites fuse together at some intermediate location between the first and second ventricular sites, and such ventricular depolarization at the second ventricular site resulting from the coupled pacing pulses.

20. The method of claim 18, wherein the first ventricular site is located in one ventricle and the second ventricular site is located in the other ventricle.
